(19) United States
(12) Patent Application Publication
(10) Pub. No.: US 2019/0352649 A1
(43) Pub. Date: Nov. 21, 2019

(54) INCREASE OF GINSENOSIDE PRODUCTION BY IMPROVEMENT OF NADPH-RELATED BIOSYNTHETIC PATHWAY IN YEAST

(71) Applicant: Intelligent Synthetic Biology Center, Daejeon (KR)

(72) Inventors: Sun-Chang Kim, Daejeon (KR); Ju Young Lee, Gyeonggi-do (KR); Jae Eung Kim, Gyeongsangbuk-do (KR); In Seung Jang, Busan (KR); Jong Geon Jegal, Ulsan (KR)

(21) Appl. No.: 16/176,220

(22) Filed: Oct. 31, 2018

(30) Foreign Application Priority Data
May 16, 2018 (KR) 10-2018-0056159

(51) Int. Cl. C12N 15/81; C12P 33/00

(52) U.S. Cl. CPC C12N 15/81; C12P 33/00

(57) ABSTRACT

The present invention relates to yeast with an enhanced ginsenoside-producing ability prepared by changing Ald2, Ald6, Zwf1, and Zms1, a method of preparing the yeast, and a method for producing ginsenosides using the yeast.

### TECHNICAL FIELD

The present invention relates to yeast for increasing ginsenoside production, and a method for producing ginsenosides using yeast.

### BACKGROUND

Saponins are glycosides widely present in plant systems, in which the non-sugar moiety consists of various cyclic compounds. Triterpene saponins, which are saponin components contained as major physiologically active components in ginseng or red ginseng, have chemical structures different from those of saponins discovered in other plants, and thus these ginseng saponins are called ginsenosides, having the meaning of ginseng glycosides, so as to distinguish them from saponins in other plants. Ginsenosides can be classified into three types according to the structure of ginsenosides: protopanaxadiol-type (PPD-type) ginsenosides, protopanaxatriol-type (PPT-type) ginsenosides, and oleanolic acid-type ginsenosides.

Pharmacological study results of ginseng have increased public interest in ginseng saponins (i.e., ginsenosides), and thus there is a growing need for the mass production of ginsenosides. However, the mass production of useful materials of ginseng by conventional cultivation methods has problems in that it would require a long culture period of 4 to 6 years, presenting difficulties in controlling pests and diseases by shading culture, rotation culture, etc., and thus there is an urgent need for the development of a new alternative production method.

Recently, many ginseng saponin-related genes have been discovered based on bioengineering technology, and as a result, the development of techniques for mass production of ginsenosides in yeast using these genes has recently started to receive attention as well. Since ginsenosides are biosynthesized in plants through the isoprenoid biosynthetic pathways including the mevalonic acid biosynthetic pathway, synthetic biology studies have been attempted to develop ginsenoside-producing strains by redesigning the ergosterol biosynthetic pathway of yeast.

Recently, China's Huang and Zhang joint research team reported that it had succeeded in producing protopanaxadiol by expressing, in Saccharomyces cerevisiae (yeast), the protopanaxadiol dammarenediol-II synthase gene and the protopanaxadiol synthase gene of ginseng along with the NADPH-cytochrome P450 reductase gene obtained from Arabidopsis thaliana (Dai, Z. et al., (2013) Metabolic engineering of Saccharomyces cerevisiae for production of ginsenosides. Metab. Eng. 20: 146 to 156.).

In the future, it is expected that ginsenoside-producing synthetic yeast will be able to provide, through more optimized work, a cost-effective production process that can replace the complex process of extracting ginsenosides from plants.

Under the circumstances, the present inventors have made efforts to increase the amount of ginsenoside production using yeast. As a result, they have developed ginsenoside-producing yeast, where the expression levels of NADPH biosynthesis-related genes are altered, and a method for preparing the yeast, and they have confirmed that the yeast produces an increased amount of protopanaxadiol (i.e., an intermediate product of ginsenoside biosynthesis) compared to the existing yeast having the ginsenoside-producing ability, thereby completing the present invention.

### DISCLOSURE

### Technical Problem

An object of the present invention is to provide yeast for producing ginsenosides.

Another object of the present invention is to provide a method for preparing the yeast.

Still another object of the present invention is to provide a method for producing ginsenosides in high yield using the yeast.

### Technical Solution

The preferred embodiments are described in detail is as follows. Meanwhile, respective descriptions and embodiments disclosed in the present invention may also be applied to other descriptions and embodiments. That is, all combinations of various elements disclosed in the present invention fall within the scope of the present invention. Further, the scope of the present invention is not limited by the specific description below.

In order to achieve the above objects, an aspect of the present invention provides yeast for producing ginsenosides, in which the expression levels of NADPH biosynthesis-related genes are changed compared to their endogenous expression levels.

In order to achieve the above objects, another aspect of the present invention provides a composition for producing ginsenosides, containing yeast for producing ginsenosides in which the expression levels of NADPH biosynthesis-related genes are changed compared to their endogenous expression levels.

As used herein, the term “reduced nicotinamide adenine dinucleotide phosphate (NADPH)” refers to a kind of coenzyme which participates, as an electron donor, in many reactions of oxidoreductase and dehydrogenase along with NADH, which shares the structure of nicotinamide adenine dinucleotide, thereby providing reducing power. The oxides of these coenzymes (i.e., NAD⁺ and NADP⁺) are known to have an important role of receiving the energy generated in the catabolic reaction in the form of electrons and protons, and participate in the oxidation-reduction enzyme reaction as an electron receptor.

The present invention is characterized in that the ginsenoside production is increased by changing the expression levels of the NADPH biosynthesis-related genes (increased or decreased). For the purposes of the present invention, any gene among the NADPH biosynthesis-related genes involved in the mass production of ginsenosides, specifically for the production of protopanaxadiol (PPD), can be used without limitation.

In embodiments of the present invention, strains where the genes related to the NADPH biosynthetic pathway are modified were prepared, and thereby the amounts of NADPH production for wild-type yeast cells and transformed yeast cells were confirmed (Examples 1 to 3). Specifically, various kinds of strains (e.g., strains where Ald2 gene or Gdh1 gene is inactivated; strains where Gnd1 gene, Gdh2 gene, Ald6 gene, Zwf1 gene, or Stb5 gene is overexpressed; strains where Ald2 gene is inactivated and Ald6 gene is overexpressed; strains where Gdh1 gene is inactivated and Gdh2 gene is overexpressed; etc.) were prepared so as to examine whether there is any increase in the amount of NADPH production in these strains.

The NADPH biosynthesis-related gene may be at least one selected from the group consisting of Ald2, Gdh1, Gnd1, Gdh2, Ald6, Stb5, Zwf1, and Zms1 genes, more specifically at least one selected from the group consisting of Ald2, Ald6, Zwf1, and Zms1 genes, but the NADPH biosynthesis-related genes are not limited thereto. The NADPH biosynthesis-related gene may be 9 genes, 8 genes, 7 genes, 6 genes, 5 genes, 4 genes, 3 genes, 2 genes, or 1 gene.

The sequences of the genes or enzymes encoded by these genes may be obtained from a known database (e.g., NCBI, etc.), but the available sources are not limited thereto.

ALD2, which is the enzyme encoded by the Ald2 gene, is an enzyme converting acetaldehyde to acetate, and it produces NADH during the process. Additionally, ALD6 is an enzyme that performs the same reaction as ALD2 (i.e. isozyme), and likewise produces NADPH during the process. Additionally, ZMS1, which is an enzyme encoded by the Zms1 gene, is a transcription regulation factor and is known to induce overexpression of the Ald6 gene.

Specifically, the Ald2 may have a gene sequence of SEQ ID NO: 1 and the Ald6 may have a gene sequence of SEQ ID NO: 2, but any sequence encoding ALD2 and ALD6 may be included without limitation.

ZWF1, which is the enzyme encoded by the Zwf1 gene, is an essential enzyme in the pentose phosphate pathway (PP pathway), and it catalyzes the first step of the PP pathway. ZWF1 is known to be involved in oxidative stress, and to be responsible for production of pentose and NADPH in the cell.

Specifically, the Zwf1 may have a gene sequence of SEQ ID NO: 3 and Zms1 may have a gene sequence of SEQ ID NO: 4, but any sequence encoding ZWF1 and ZMS1 may be included without limitation. In addition, Gdh1, Gnd1, Gdh2, and Stb5 may have each of the gene sequences of SEQ ID NO: 31, SEQ ID NO: 32, SEQ ID NO: 33, and SEQ ID NO: 34, but any sequence encoding GDH1, GND1, GDH2, and STB5 enzymes may be included without limitation.

For the purposes of the present invention, any gene which is involved in the increase of NADPH biosynthesis, thus being capable of increasing protopanaxadiol (PPD)-based ginsenosides, may be applicable without limitation.

Additionally, in the present invention, the above genes can include not only those sequences described above, but also those genes which have a homology to the above sequences of at least 80%, specifically at least 90%, more specifically at least 95%, and even more specifically at least 99%. Additionally, it is apparent that any gene sequence which has a nucleotide sequence having a homology to the above sequences, and encodes an enzyme which exhibits effects substantially the same as or corresponding to those of the above enzymes may be included without limitation. Additionally, it is apparent that any nucleotide sequence having such a homology falls within the scope of the present invention, even if the nucleotide sequence may include deletion, modification, substitution, or addition in part of the sequence.

In the above, the term “homology” refers to the degree of similarity to a given nucleotide sequence, and it may be expressed as a percentage (%). In the present specification, a homologous sequence having an activity the same as or similar to that of a given nucleotide sequence may be expressed as “% homology”. For example, the homology may be identified using standard software, specifically BLAST 2.0, for calculating parameters (e.g., score, identity, similarity, etc.) or by comparing sequences through hybridization experiments under defined stringent conditions. Appropriate hybridization conditions may be determined within the scope of the art and by methods well known to those skilled in the art (e.g., J. Sambrook et al., Molecular Cloning, A Laboratory Manual, 2nd Edition, Cold Spring Harbor Laboratory press, Cold Spring Harbor, New York, 1989; F. M. Ausubel et al., Current Protocols in Molecular Biology, John Wiley & Sons, Inc., New York).

The yeast of the present invention is characterized in that the expression levels of the NADPH biosynthesis-related proteins are changed compared to their endogenous levels.

As used herein, the term “endogenous expression level” refers to the expression level of mRNA or protein of a microorganism that is expressed in a natural state or in its parent strain before the expression level of the corresponding protein undergoes modification. This is essentially the degree to which a given mRNA or protein is produced in a cell or tissue of a strain, under normal circumstances or prior to controlling the expression of a specific protein. The endogenous expression levels can be compared between strain types, cell types, and tissues, or may be compared to expression levels induced by some stimuli. Specifically, the endogenous expression level may be an expression level of mRNA or a protein expressed in a microorganism where the expression of NADPH biosynthesis-related proteins are not regulated.

The above term “change” may be used to include the increase or decrease or both. For the purposes of the present invention, the term “change” may include decreasing the expression of a particular gene by inactivating the gene and increasing the expression of a particular gene by overexpressing the gene. That is, “decrease” may be used to refer to “decrease in expression level compared to its endogenous expression level” and “increase” may be used to refer to “increase in expression level compared to its endogenous expression level”.

Specifically, the phrase “increase in expression level compared to its endogenous expression level” means that the gene encoding the corresponding polypeptide is expressed at a higher level compared to when the gene is in a natural state or before modification, and thus the corresponding polypeptide with functional ability is produced in a large amount.

Specifically, the increase in expression level compared to its endogenous expression level may be performed by the following methods:

1) a method for increasing the copy number of the polynucleotide that encodes the protein,

2) a method for modifying the expression control sequence so as to increase the expression of the polynucleotide,

3) a method for modifying the polynucleotide sequence on the chromosome so as to enhance the activity of the protein, or

4) a method for modification so as to enhance the activity of the protein by a combination of the above methods 1) to 3), etc., but the methods are not limited thereto.

The method of increasing the copy number of the polynucleotide in method 1) may be performed in the form where the polynucleotide is operably linked to a vector, or by inserting the polynucleotide into the chromosome in a host cell, but the method is not particularly limited thereto. Specifically, it may be performed by inserting a vector into a host cell, in which the vector is operably linked to a polynucleotide encoding the protein of the present invention, and can replicate and function regardless of the host cell. Alternatively, it may be performed by a method for increasing the copy number of the polynucleotide in the chromosome in the host cell by inserting a vector into the host cell, in which the vector is operably linked to the polynucleotide and is able to insert the polynucleotide into the chromosome in the host cell. The introduction may be performed by those skilled in the art by appropriately selecting a known transformation method, and the enzyme may be produced by the expression of the introduced polynucleotide in a host cell, and thereby its activity can be increased.

Next, the method 2) of modifying the expression control sequence so as to increase the expression of the polynucleotide may be performed by inducing a mutation on the sequence by deletion, insertion, or non-conservative or conservative substitution of a nucleic acid sequence, or a combination thereof, such that the activity of the expression control sequence can be further enhanced, or by replacing the sequence with a nucleic acid sequence having enhanced activity, but the method is not particularly limited thereto. The expression control sequence may include a promoter, an operator sequence, a sequence encoding a ribosomal binding site, a sequence controlling the termination of transcription and translation, etc., but the method is not particularly limited thereto.

In addition, the method 3) for modifying the polynucleotide sequence on the chromosome may be performed by inducing a mutation on the sequence by deletion, insertion, or non-conservative or conservative substitution of a nucleic acid sequence, or a combination thereof, such that the activity of the polynucleotide sequence can be further enhanced, or by replacing the sequence with a polynucleotide sequence having enhanced activity, but the method is not particularly limited thereto.

Finally, the method 4) of modifying so as to enhance the activity of the protein by a combination of the above methods 1) to 3) may be performed by applying at least one method among a method of increasing the copy number of the polynucleotide that encodes the protein; a method of modifying the expression control sequence so as to increase the expression of the polynucleotide; a method for modifying the polynucleotide sequence on the chromosome; and a method for modifying an exogenous polynucleotide exhibiting the activity of the enzyme, or modifying a codon-optimized modified polynucleotide thereof.

As used herein, the term “vector” refers to a DNA construct including the nucleotide sequence of the polynucleotide encoding a target protein, in which the target protein is operably linked to a suitable control sequence so that the target protein can be expressed in an appropriate host. The control sequence may include a promoter capable of initiating transcription, any operator sequence capable of controlling such transcription, a sequence encoding an appropriate mRNA ribosome-binding domain, and a sequence for controlling the termination of transcription and translation, but the control sequence is not limited thereto. The vector, after being transformed into a suitable host cell, may be replicated or function irrespective of the host genome, or may be integrated to the genome itself.

The vector used in the present invention may not be particularly limited as long as the vector is replicable in the host cell, and any vector known in the art may be used. Examples of the vector conventionally used may include natural or recombinant plasmids, and the replication origin may include the autonomous replication sequence (ARS) of yeast. The autonomous replication sequence may be stabilized by the centrometric sequence (CEN) of yeast. The promoter may be one selected from the group consisting of CYC promoter, TEF promoter, GPD promoter, PGK promoter, ADH promoter, etc. The terminator may be selected from the group consisting of PGK1, CYC1, GAL1, etc. The vector may further include a selection marker.

The vector used in the present invention is not particularly limited, but any known expression vector may be used. Additionally, a polynucleotide encoding a target protein may be inserted into the chromosome in a cell using a vector for chromosomal insertion. The insertion of the polynucleotide into the chromosome may be performed using any method known in the art, e.g., by homologous recombination, but the insertion is not limited thereto. A selection marker for confirming the insertion of the vector into the chromosome may be further included. The selection marker is used for selection of cells transformed with the vector, i.e., in order to confirm whether the target nucleic acid molecule has been inserted, and markers capable of providing selectable phenotypes such as drug resistance, auxotrophy, resistance to cytotoxic agents, and expression of surface proteins may be used. Under the circumstances where selective agents are treated, only the cells capable of expressing the selection markers can survive or express other phenotypic traits, and thus the transformed cells can easily be selected.

As used herein, the term “transformation” refers to a process of introducing a vector, which includes a target protein-encoding polynucleotide, into a host cell such that the protein encoded by the polynucleotide can be expressed in the host cell. It does not matter whether the transformed polynucleotide is inserted into the chromosome of the host cell and located thereon or located outside of the chromosome, as long as the transformed polynucleotide can be expressed in the host cell. Additionally, the polynucleotide may include DNA and RNA encoding the target protein. The polynucleotide may be introduced in any form, as long as the polynucleotide can be introduced into the host cell and expressed therein. For example, the polynucleotide may be introduced into the host cell in the form of an expression cassette, which is a gene construct including all elements required for its autonomous expression. The expression cassette may include a promoter, a transcription termination signal, a ribosome binding site, and a translation termination signal that are operably linked to the polynucleotide. The expression cassette may be in a form of an expression vector capable of self-replication. Further, the polynucleotide may be introduced into the host cell as is to be operably linked to the sequence required for its expression in the host cell, but the form of the expression cassette is not limited thereto.

The transformation method may include any method which can introduce nucleic acids into a cell, and the transformation may be performed by selecting an appropriate technique as known in the art according to the host cell. For example, the method may include electroporation, calcium phosphate (CaPO₄) precipitation, calcium chloride (CaCl₂) precipitation, microinjection, a polyethylene glycol (PEG) method, a DEAE-dextran method, a cationic liposome method, and a lithium acetate/DMSO method, etc., but the method is not limited thereto.

Additionally, as used herein, the term “operably linked” refers to a functional linkage between a promoter sequence, which initiates and mediates the transcription of the polynucleotide encoding the target protein of the present invention, and the polynucleotide sequence. The operable linkage may be prepared by genetic recombination technology known in the art, and site-specific DNA cleavage and linkage may easily be performed using enzymes, etc., known in the art, but the method is not limited thereto.

As used herein, the term “decrease in expression level compared to its endogenous expression level” refers to inactivation of a particular gene, and specifically, “inactivation” refers to a case where the activity of an enzyme protein originally possessed by a microorganism is weakened compared to its endogenous activity or the activity before modification of the protein, the protein is not expressed, or the protein is expressed but exhibits no activity. Additionally, in the present invention, the term inactivation may be used to refer to all of decrease, deletion, and weakening.

Specifically, the inactivation refers to a concept including a case where the activity of the enzyme itself is weakened compared to that originally possessed by a microorganism due to a modification in the enzyme-encoding polynucleotide, etc., or removed; a case where the level of overall enzyme activity is lower than that of the wild-type strain of the microorganism due to inhibition of expression or inhibition of translation of the gene encoding the enzyme, etc., or removed; a case where all or part of the gene is deleted; and a combination thereof, but the inactivation is not limited thereto.

The inactivation of an enzyme may be achieved by applying various methods well known in the art. Examples of the methods may include 1) a method of substituting the enzyme-encoding gene on the chromosome with a gene mutated to reduce the activity of the enzyme, including the case where the enzyme activity is removed; 2) a method of modifying the expression control sequence of the enzyme-encoding gene on the chromosome; 3) a method of substituting the expression control sequence of the enzyme-encoding gene with a sequence having weak or no activity; 4) a method of deleting all or part of the enzyme-encoding gene on the chromosome; 5) a method of introducing an antisense oligonucleotide (e.g., antisense RNA) which binds complementary to a transcript of the gene on the chromosome, thereby inhibiting the translation from the mRNA into the enzyme; 6) a method of artificially incorporating a complementary sequence to the SD sequence into the upstream of the SD sequence of the enzyme-encoding gene, forming a secondary structure, thereby making the attachment of ribosome thereto impossible; 7) a method of incorporating a promoter to the 3′ terminus of the open reading frame (ORF) of the corresponding sequence to be transcribed in reverse (reverse transcription engineering (RTE)), etc., and also a combination thereof, but the methods are not particularly not limited thereto.

The gene sequence on the chromosome may be modified by inducing modification in the sequence by deletion, insertion, non-conservative or conservative substitution, or a combination thereof in the gene sequence for further weakening the enzyme activity; or by substituting with a gene sequence which was improved to have weaker activity or a gene sequence which was improved to have no activity, but the method is not limited thereto.

The expression control sequence may be modified by inducing modification in the expression control sequence by deletion, insertion, non-conservative or conservative substitution, or a combination thereof of its nucleic acid sequence so as to further weaken the activity of the expression control sequence; or by substituting with a nucleic acid sequence having much weaker activity. The expression control sequence may include a promoter, an operator sequence, a sequence encoding a ribosome-binding region, and sequences controlling the termination of transcription and translation, but is not limited thereto.

Additionally, the method of deleting all or part of a polynucleotide encoding an enzyme may be performed by substituting the polynucleotide encoding the endogenous target protein within the chromosome with a polynucleotide or marker gene having partial deletion in the nucleic acid sequence using a vector for chromosomal insertion within a cell. In an exemplary embodiment of the method of deleting all or part of a polynucleotide, a method for deleting a polynucleotide by homologous recombination may be used, but the method is not limited thereto.

In a case where the polynucleotide is an aggregate of polynucleotides that can exhibit a function, it may be described as a gene. In the present invention, the term polynucleotide may be used interchangeably with gene.

In the above, the term “part” may vary depending on the kinds of polynucleotides, and it may specifically refer to 1 to 300, more specifically 1 to 100, and even more specifically 1 to 50, but the term is not particularly limited thereto.

In an embodiment of the present invention, strains with an increased concentration of NADPH compared to the control group were selected, and then a yeast strain where Ald2 gene is inactivated and Ald6 gene is overexpressed in a PPD yeast cell (PPD,Δald2::ald6) was prepared from the selected strains so as to confirm whether the genes which affect the NADPH biosynthetic pathway can also affect the growth of PPD yeast cells and PPD production (Example 4). Additionally, a strain where Zwf1 is further inactivated in the above strain (PPD,Δald2::ald6,Δzwf1) and a strain where Zms1 is further overexpressed in the above strain (PPD,Δald2::ald6,p416_GPD_Zms1) were prepared.

As a result, it was confirmed that the prepared transformed yeast can exhibit higher protopanaxadiol (PPD) productivity compared to the control group. Specifically, it was confirmed that the strain where Ald2 gene is inactivated and Ald6 gene is overexpressed can produce about a 4-fold higher protopanaxadiol production compared to the control group, and that in cases where the Zwf1 gene is further inactivated or the Zms1 gene is further overexpressed, where the Zwf1 gene and the Zms1 gene affect the NADPH biosynthetic pathway, the amount of protopanaxadiol production was significantly increased (Table 5 and FIGS. 7 and 8).

From the above results, showing that when the expression levels of the NADPH biosynthesis-related genes (Ald2, Ald6, Zwf1, and Zms1) were changed by the endogenous expression levels, the amount of production of protopanaxadiol, which is an intermediate product of ginsenoside biosynthesis, was increased, it is expected that the ginsenoside-producing ability of these genes will be improved.

As used herein, the term “yeast for producing ginsenosides” refers to yeast which naturally has a ginsenoside-producing ability; or yeast which does not have a ginsenoside-producing ability in its parent strain but a ginsenoside-producing ability is provided thereto.

Specifically, in the present invention, a ginsenoside-producing microorganism may refer to the wild-type microorganism itself; a microorganism to which a gene related to the external ginsenoside biosynthesis is introduced, thus having a ginsenoside-producing ability; or a microorganism in which a gene related to the mechanism of external NADPH production is inserted, the activity of an endogenous gene thereof is enhanced or inactivated, and its ginsenoside-producing ability is thereby increased.

More specifically, the yeast may be one that belongs to the genus Saccharomyces, the genus Zygosaccharomyces, the genus Pichia, the genus Kluyveromyces, the genus Candida, the genus Shizosaccharomyces, the genus Issachenkia, the genus Yarrowia, or the genus Hansenula.

The yeast belonging to the genus Saccharomyces may be, for example, S. cerevisiae, S. bayanus, S. boulardii, S. bulderi, S. cariocanus, S. cariocus, S. chevaliers, S. dairenensis, S. ellipsoideus, S. eubayanus, S. exiguus, S. florentinus, S. kluyveri, S. martiniae, S. monacensis, S. norbensis, S. paradoxus, S. pastorianus, S. spencerorum, S. turicensis, S. unisporus, S. uvarum, or S. zonatus. More specifically, the yeast may be Saccharomyces cerevisiae (S. cerevisiae), but the yeast is not limited thereto.

The ginsenoside-producing microorganism may be that where the expression level of Ald2 gene is decreased compared to its endogenous expression level while the expression level of Ald6 gene is increased compared to its endogenous expression, and additionally, that where the expression level of Zwf1 gene is decreased compared to its endogenous expression, but the microorganism is not particularly limited thereto. Additionally, the microorganism capable of producing ginsenosides may be that where the expression level of Zms1 gene is further increased compared to its endogenous expression, but the microorganism is not limited thereto.

Additionally, the ginsenoside-producing microorganism may be one in which i) HMG-CoA reductase (HMG1), which converts HMG-CoA to mevalonic acid, and ii) Panax ginseng squalene epoxidase (PgSE), for enhancing the mevalonic acid metabolic pathway to increase the biosynthesis of squalene (i.e., an essential precursor for ginsenoside biosynthesis), are modified such that their activities are greater than their endogenous activities, respectively; and iii) Panax ginseng dammarenediol-II synthase (PgDDS), which converts 2,3-oxidosqualene to dammarenediol-II, iv) Panax ginseng cytochrome P450 CYP716A47 (PgPPDS), which converts dammarenediol-II to protopanaxadiol, and v) Panax ginseng NADPH-cytochrome P450 reductase (Panax PgCPR) are modified such that their activities can be introduced, but the modification may not be particularly limited thereto.

As used herein, the term “ginsenoside” refers to a dammarane-type saponin or a derivative thereof, and it has a chemical structure different from those of saponins discovered in other plants. Specifically, ginsenosides may be classified into three different groups based on their aglycone structures: protopanaxadiol (PPD)-type ginsenosides, protopanaxatriol (PPT)-type ginsenosides, and oleanolic acid-type ginsenosides. These three groups may be further classified based on the position and number of sugar moieties attached to the C-3, C-6, and C-20 positions of the rings in the aglycone structure of the compounds by a glycosidic bond. PPDs and PPTs have different hydroxylation patterns. The basic backbone of the oleanolic acid-type ginsenosides is 5-cyclic, and its aglycone is oleanolic acid; ginsenoside Ro is uniquely present in this group. At present, more than 40 kinds of ginsenosides have been isolated, and most are PPD-type ginsenosides. PPD-type ginsenosides include Rb1, Rb2, Rb3, Rc, Rd, gypenoside XVII, Compound O, Compound Mc1, F2, Compound Y, Compound Mc, Rg3, Rh2, and C-K. PPT-type ginsenosides include Re, Rg1, Rf, Rg2, Rh1, etc.

In an embodiment, the ginsenosides may be PPD-type ginsenosides, PPT-type ginsenosides, etc.; in another embodiment, the PPD, PPT, Ra3, Rb1, Rb2, Rb3, Rc, Rd, Re, Rg1, Rg2, Rg3, Rh1, Rh2, Rs1, C—O, C—Y, C-Mc1, C-Mc, F1, F2, Compound K, Gypenoside XVII, Gypenoside LXXV, Rs2, PPD, Re, Rg1, Rf, F1, Rg2, PPT, Rh1, etc. may be used alone or as a mixture; and in still another embodiment, PPD, PPT, compound K, Rb1, Rb2, Rb3, Rc, Rd, Re, F1, F2, Rg1, Rg2, Rg3, Rh1, Rh2, etc. may be used alone or as a mixture, and but the ginsenosides are not particularly limited thereto. Specifically, the ginsenosides may be protopanaxadiol-type ginsenosides.

Additionally, the yeast of the present invention may be that where the NADPH-producing ability is increased compared to its endogenous production level.

In another specific embodiment, the present invention provides yeast in which the expression level of the gene involved in the synthesis of ginsenosides is further increased compared to its endogenous expression level.

In still another specific embodiment, the present invention provides yeast in which the gene is at least one selected from the group consisting of Panax ginseng dammarenediol-II synthase (PgDDS), Panax ginseng cytochrome P450 CYP716A47 (PgPPDS), Panax ginseng NADPH-cytochrome P450 reductase (PgCPR), S. cerevisiae HMG-CoA reductase (tHMG1), and Panax ginseng squalene epoxidase (PgSE).

The enzymes related to the ginsenoside biosynthesis metabolic pathway may each have an amino acid sequence having a homology to the amino acid sequences of SEQ ID NOS: 5 to 9, of at least 70%, more specifically at least 80%, and even more specifically at least 90%.

Another aspect of the present invention provides a method for preparing yeast with an improved ginsenoside-producing ability, which includes changing the expression levels of NADPH biosynthesis-related genes compared to their endogenous expression levels.

In a specific embodiment, the present invention provides a method for preparing yeast, in which the expression level of at least one gene selected from the group consisting of Ald2, Ald6, Zwf1, and Zms1 genes, which are NADPH biosynthesis-related genes, is increased compared to its endogenous expression level.

The ginsenoside-producing yeast strain, NADPH biosynthesis-related genes, Ald2, Ald6, Zwf1, and Zms1 genes, endogenous expression level, etc. are the same as described above.

In another specific embodiment of the present invention, the ginsenoside-producing yeast strain may be that where the expression level of at least one gene, which is selected from the group consisting of Panax ginseng dammarenediol-II synthase (PgDDS), Panax ginseng cytochrome P450 CYP716A47 (PgPPDS), Panax ginseng NADPH-cytochrome P450 reductase (PgCPR), S. cerevisiae HMG-CoA reductase (tHMG1), and Panax ginseng squalene epoxidase (PgSE), is increased compared to its endogenous expression level.

Still another aspect of the present invention provides a method for producing ginsenosides, which includes culturing the yeast in a medium; and recovering ginsenosides from the yeast or the medium.

In the above method, the cultivation of the yeast may be performed by a known batch culture, continuous culture, fed-batch culture, etc., but the cultivation is not particularly limited thereto. In particular, the culture conditions are not particularly limited, but an optimal pH (e.g., pH 5 to pH 9, specifically pH 6 to pH 8, and most specifically pH 6.8) may be adjusted using a basic compound (e.g., sodium hydroxide, potassium hydroxide, or ammonia) or an acidic compound (e.g., phosphoric acid or sulfuric acid). An aerobic condition may be maintained by adding oxygen or an oxygen-containing gas mixture to the culture. The culture temperature may be maintained at 20° C. to 45° C., and specifically at 25° C. to 40° C., and the culturing may be performed for about 10 hours to about 160 hours, but is not limited thereto. The ginsenosides produced by the cultivation may be secreted into the medium or may remain within the cells.

Further, in the culture medium to be used, as a carbon source, sugars and carbohydrates (e.g., glucose, sucrose, lactose, fructose, maltose, molasses, starch, and cellulose), oils and fats (e.g., soybean oil, sunflower seed oil, peanut oil, and coconut oil), fatty acids (e.g., palmitic acid, stearic acid, and linoleic acid), alcohols (e.g., glycerol and ethanol), organic acids (e.g., acetic acid), etc. may be used alone or in combination, but the carbon source is not limited thereto. As a nitrogen source, a nitrogen-containing organic compound (e.g., peptone, a yeast extract, a meat extract, a malt extract, a corn steep liquor, soybean meal, and urea) or an inorganic compound (e.g., ammonium sulfate, ammonium chloride, ammonium phosphate, ammonium carbonate, and ammonium nitrate), etc. may be used alone or in combination, but the nitrogen source is not limited thereto. As a phosphorus source, potassium dihydrogen phosphate, dipotassium hydrogen phosphate, a sodium-containing salt corresponding thereto, etc. may be used alone or in combination, but the phosphorus source is not limited thereto. The medium may also include essential growth-promoting materials such as other metal salts (e.g., magnesium sulfate or iron sulfate), amino acids, and vitamins.

The above method may further include recovering produced ginsenosides. This recovery step may be a step of recovery from the cultured cells or a supernatant thereof, and an appropriate process for recovery may be selected by those skilled in the art.

With regard to the method of recovering the ginsenosides produced during the cultivation of the present invention, the target products may be collected from the culture using an appropriate method known in the art according to the cultivation method (e.g., batch culture, continuous culture, fed-batch culture, etc.). For example, centrifugation, filtration, anion exchange chromatography, crystallization, HPLC, etc. may be used, and the desired ginsenoside may be recovered from the medium or microorganism using an appropriate method known in the art. The method of recovering ginsenosides may further include a step of purification.

### Advantageous Effects of the Invention

The ginsenoside-producing yeast of the present invention, in which the expression levels of the NADPH biosynthesis-related genes are changed, has an effect of increasing the amount of protopanaxadiol production (i.e., an intermediate product of ginsenoside biosynthesis) compared to the existing yeast with a ginsenoside-producing ability.

### BRIEF DESCRIPTION OF DRAWINGS

FIG. 1 is a drawing briefly illustrating the increase of ginsenoside production by the enhancement of the NADPH biosynthetic pathway.

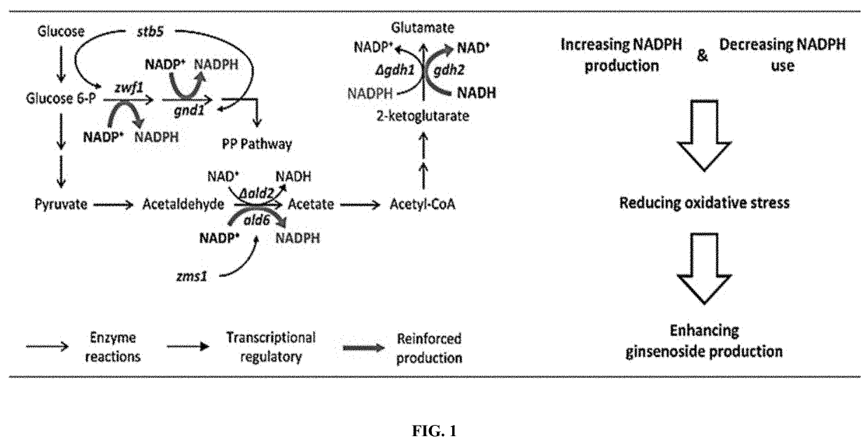

FIG. 2 is a drawing illustrating a metabolic pathway of ginsenoside biosynthesis.

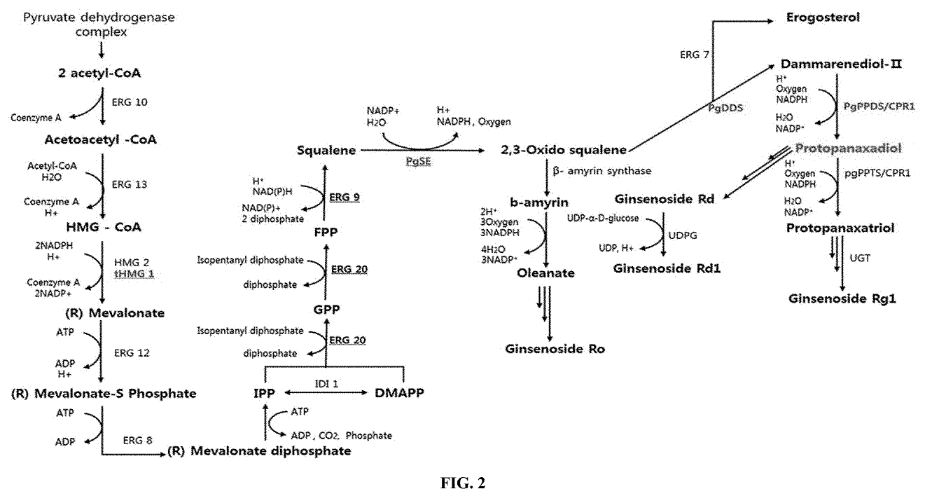

FIG. 3 is a drawing illustrating a vector map of pUC57-URA3Myc, a vector prepared for the inactivation of Ald2 gene or Gdh1 gene.

FIG. 4 is a drawing illustrating a vector map of p416_GPD, a vector prepared for the overexpression of Gnd1 gene, Gdh2 gene, Ald6 gene, Zwf1 gene, or Stb5 gene.

FIG. 5 is a drawing illustrating a vector map of pUC57GPD-URA3Myc, a vector prepared for the inactivation ofAld2 gene and overexpression ofAld6 gene; and for the inactivation of Gdh1 gene and overexpression of Gdh2 gene.

FIG. 6 is a graph illustrating the level of NADPH production in a wild-type yeast cell and a transformed yeast cell, in which C represents S. cerevisiae CEN.PK2-1D as a control strain; -Ald2 represents CEN.PK2-1D,Δald2; −Gdh1 represents CEN.PK2-1D,ΔGdh1; +GND1 represents CEN.PK2-1D,p416_GPD_GND1; +Gdh2 represents CEN.PK2-1D,p416_GPD_Gdh2; +Ald6 represents CEN.PK2-1D,p416_GPD_Ald6; +Zwf1 represents CEN.PK2-1D, p416_GPD_ Zwf1; +STB5 represents CEN.PK2-1D,p416_GPD_STB5; −Ald2/+Ald6 represents CEN.PK2-1D,Δald2::ald6; and −Gdh1/+Gdh2 represents CEN.PK2-1D,ΔGdh1::Gdh2.

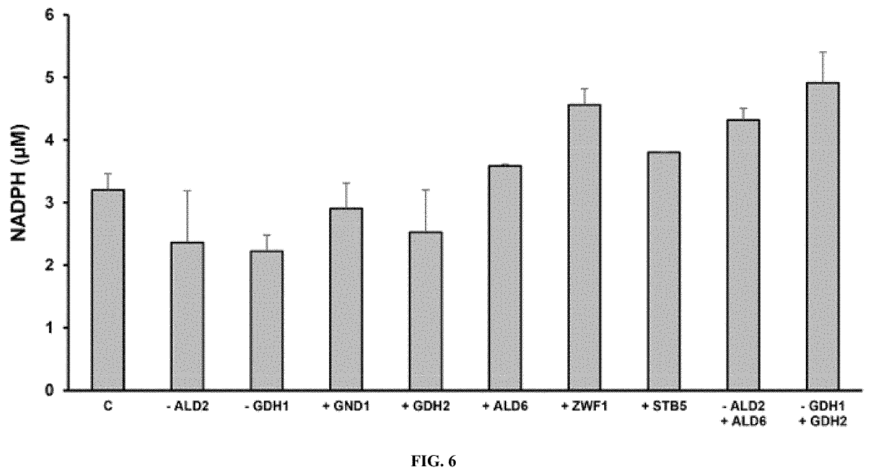

FIG. 7 is a graph illustrating the level of PPD production in a transformed yeast cell, in which Control represents a PPD strain (S. cerevisiae CEN.PK2-1D Δtrp1::PGPD1 tHMG1+PGPD1 PgSE Δleu2::PGPD1 PgDDS+PGPD1 PgPPDS+PGPD1 PgCPR); +Zwf1 represents PPD,p416_GPD_Zwf1; +STB5 represents PPD,p416_GPD_Stb5; −Ald2/+Ald6 represents PPD,Δald2::ald6; and −Gdh1/+Gdh2 represents PPD,ΔGdh1::Gdh2.

FIG. 8 is a graph illustrating the level of PPD production in a transformed yeast cell in terms of relative value, in which Control represents a PPD strain (S. cerevisiae CEN.PK2-1D Δtrp1::PGPD1 tHMG1+PGPD1 PgSE Δleu2::PGPD1 PgDDS+PGPD1 PgPPDS+PGPD1 PgCPR); +Zms1 represents PPD,p416_GPD_Zms1; −Ald2/+Ald6 represents PPD,Δald2::ald6; −Ald2/+Ald6/−Zwf1 represents PPD,Δald2::ald6,ΔZwf1; and −Ald2/+Ald6/+Zms1 represents PPD,Δald2::ald6,p416_GPD Zms1. Each value represents the change in the fold of protopanaxadiol produced in each prepared strain when the production concentration of protopanaxadiol at time 72 hours is set at 1.

### DETAILED DESCRIPTION OF THE INVENTION

Hereinafter, the present invention will be described in more detail with reference to the following Examples and Experimental Examples. However, these Examples and Experimental Examples are for illustrative purposes only and the scope of the invention is not limited by these Examples and Experimental Examples.

### EXAMPLE 1

### Preparation of PPD Modified Yeast Strain

A protopanaxadiol (PPD)-producing yeast strain of the present invention was prepared by i) introducing a ginsenoside biosynthesis metabolic pathway into Saccharomyces cerevisiae (S. cerevisiae) CEN.PK2-1D wild-type strain [(MATα ura3-52; trp1-289; leu2-3,112; his3Δ 1; MAL2-8; SUC2) and ii) enhancing the mevalonic acid metabolic pathway, which increases biosynthesis of squalene (i.e., an essential precursor for ginsenoside biosynthesis) of the strain, and the prepared strain was named as PPD modified yeast strain(PPD strain).

The genotype of the PPD strain is S. cerevisiae CEN.PK2-1D Δtrp1::P_{GPD1} tHMG1+P_{GPDI} PgSE+Δleu2::P_{GPD1} PgDDS+P_{GPDI} PgPPDS+P_{GPDI} PgCPR.

Genes, which encode ginsenoside biosynthesis enzymes, i.e. Panax ginseng dammarenediol-II synthase (PgDDS, SEQ ID NO: 5), Panax ginseng cytochrome P450 CYP716A47 (PgPPDS, SEQ ID NO: 6), and Panax ginseng NADPH-cytochrome P450 reductase (PgCPR, SEQ ID NO: 7); and metabolic pathway enzymes for enhancing the mevalonic acid metabolic pathway tHMG1(S.cerevisiae HMG-CoA reductase, SEQ ID NO: 8) and Panax ginseng squalene epoxidase, (PgSE, SEQ ID NO: 9), were each transcribed from GPD1(TDH3), a constitutive high-expression promoter, and expressed.

### Example 2

### Preparation of Modified Strains Related to NADPH Biosynthetic Pathway

### 2-1: Preparation of Strain Where Ald2 Gene or Gdh1 Gene is Inactivated

To examine whether the inactivation of the Ald2 gene or Gdh1 gene, which are genes related to NADPH biosynthesis or consumption pathway, in the PPD modified yeast strain may be involved in the growth of the above yeast strain and PPD-producing ability, modified yeast strains where the Ald2 gene or Gdh1 gene was inactivated were prepared.

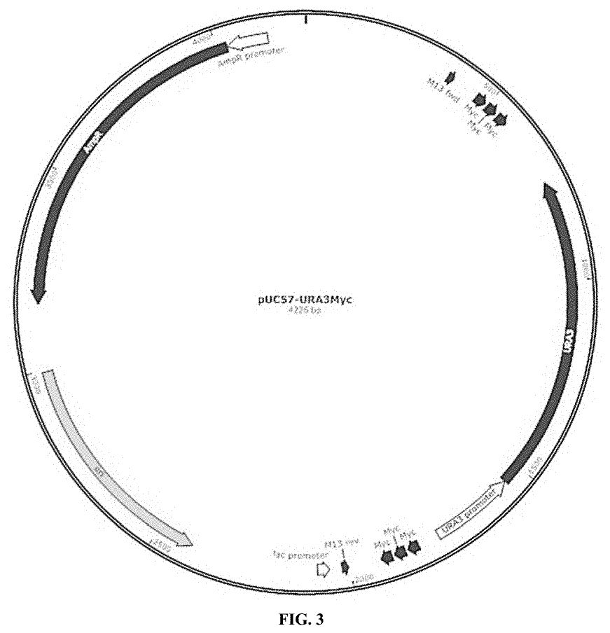

To inactivate the Ald2 gene on the genome, a cassette that inactivates the Ald2 gene was prepared by performing PCR using the previously prepared pUC57-URA3Myc vector (Ju Young Lee et al., (2015) Biotechnol. Bioeng., 112, 751-758.) as a template (FIG. 3 and SEQ ID NO: 18) and Del_Ald2_F and Del_Ald2_R, the homologous recombinant sequences of Ald2 gene region on the genome, as primers. The prepared inactivation cassette was transformed into the cells of each wild-type yeast strain of S. cerevisiae CEN.PK2-1D. The transformation was performed by conventional heat shock transformation. After the transformation, the cells were cultured in a uracil drop-out medium so that the Ald2 gene on the genome was able to be substituted with the cassette containing URA3.

The inactivity of the Ald2 gene in the obtained strain was confirmed by performing PCR using the genomic DNA of the above cells as a template and a primer set of Ald2_conf_F and Ald2_conf_R, and the strain was named as CEN.PK2-1D, Δald2. In the same manner, a Gdh1 inactivation cassette was prepared using a primer set of Del_Gdh1_F and Del_Gdh1_R, and the inactivity of the Gdh1 gene was confirmed using a primer set of Gdh1_conf_F and Gdh1_conf_R primers, and the strain was named as a CEN.PK2-1D, ΔGdh1. The primers used above are shown in Table 1 below.

TABLE 1

| | | SEQ |
|---|---|---|
| | | ID |
| Name | Primer Sequence (5′→3′) | NO |
| Del_Ald2_F | TTACATTGCATGTCCATCAAAAACAAT | 10 |
| | CGTGAAAATAAGCCAAAAGAAAACCAG | |
| | TCACGACGTTGTAAAA | |
| Del_Ald2_R | CTGCAACATCCCACTCCTTCTTTGCAG | 11 |
| | TTTCTTTAAACTTTTCAACAAACAGGT | |
| | TTCCCGACTGGAAAGC | |
| Ald2_conf_F | TTACATTGCATGTCCATCAAAAACA | 12 |
| Ald2_conf_R | CTGCAACATCCCACTCCTTC | 13 |
| Del_Gdh1_F | ACTATCGCATTATTCTAATATAACAGT | 14 |
| | TAGGAGACCAAAAAGAAAAAGAACCAG | |
| | TCACGACGTTGTAAAA | |
| Del_Gdh1_R | GACGGCAATAGCTTCTGGAGTGGAACC | 15 |
| | CATGTTGGAACCTTCGGCAATAAAGGT | |
| | TTCCCGACTGGAAAGC | |
| Gdh1_conf_F | CAGTTAGGAGACCAAAAAGAAAAAGAA | 16 |
| Gdh1_conf_R | GACGGCAATAGCTTCTGGAG | 17 |

### 2-2: Preparation of Strain Overexpressing Gnd1, Gdh2, Ald6, Zwf1, or Stb5

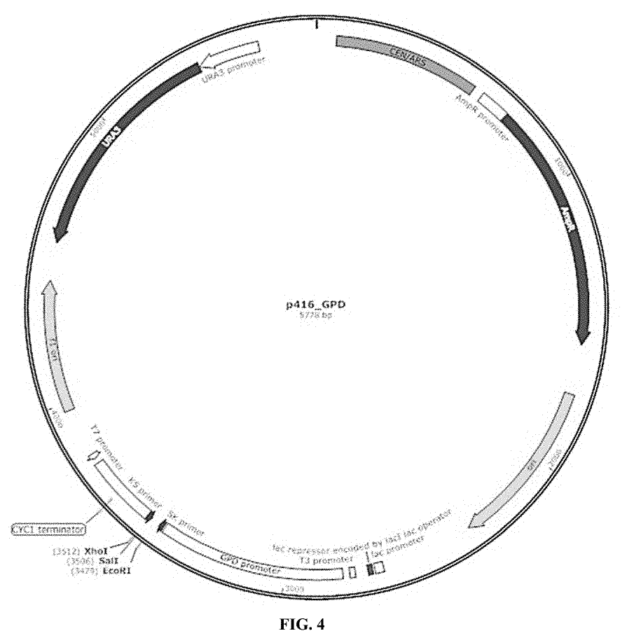

For the overexpression of the Gnd1 gene, the Gnd1 gene was amplified from the genomic DNA of S. cerevisiae CEN.PK2-1D by performing PCR using a primer set of Gnd1_F and Gnd1_R, and the amplified products were digested with EcoRI and XhoI, and ligated to the p416_GPD vector (FIG. 4 and SEQ ID NO: 29), which was also digested with EcoRI and XhoI, and thereby the p416_GPD_Gnd1 vector was prepared.

For the overexpression of the Gdh2 gene, the Gdh2 gene was amplified from the genomic DNA of S. cerevisiae CEN.PK2-1D by performing PCR using a primer set of Gdh2_F and Gdh2_R, and the amplified products were digested with XbaI and SmaI, and ligated to the p416_GPD vector, which was also digested with XbaI and SmaI, and thereby the p416_GPD_Gdh2 vector was prepared.

For the overexpression of the Ald6 gene, the Ald6 gene was amplified from the genomic DNA of S. cerevisiae CEN.PK2-1D by performing PCR using a primer set of Ald6_F and Ald6_R, and the amplified products were digested with BamHI and XhoI, and ligated to the p416_GPD vector, which was also digested with BamHI and XhoI, and thereby the p416_GPD_Ald vector was prepared.

For the overexpression of the Zwf1 gene, the Zwf1 gene was amplified from the genomic DNA of S. cerevisiae CEN.PK2-1D by performing PCR using a primer set of Zwf1_F and Zwf1_R, and the amplified products were digested with EcoRI and XhoI, and ligated to the p416_GPD vector, which was also digested with EcoRI and XhoI, and thereby the p416_GPD_Zwf1 vector was prepared.

For the overexpression of the Stb5 gene, the Stb5 gene was amplified from the genomic DNA of S. cerevisiae CEN.PK2-1D by performing PCR using a primer set of Stb5_F and Stb5_R, and the amplified products were digested with EcoRI and SalI, and ligated to the p416_GPD vector, which was also digested with EcoRI and SalI, and thereby the p416_GPD_Stb5 vector was prepared.

The primers used above are shown in Table 2 below.

TABLE 2

| | | SEQ |
|---|---|---|
| | | ID |
| Name | Primer Sequence (5′→3′) | NO |
| Gnd1_F | GGAATTCATGTCTGCTGATTTCGGTTT | 19 |
| Gnd1_R | CCGCTCGAGTTAAGCTTGGTATGTAGAGGAAGAA | 20 |
| Gdh2_F | GCTCTAGAATGCTTTTTGATAACAAAAATCGCGG | 21 |
| Gdh2_R | TCCCCCGGGTCAAGCACTTGCCTCCGCTT | 22 |
| Ald6_F | CGGGATCCATGACTAAGCTACACTTTGACACTGC | 23 |
| Ald6_R | CCGCTCGAGTTACAACTTAATTCTGACAGCTTTT | 24 |
| | ACTTCAG | |
| Zwfl_F | GGAATTCATGAGTGAAGGCCCCGTCAA | 25 |
| Zwfl_R | CCGCTCGAGCTAATTATCCTTCGTATCTTCTGGC | 26 |
| Stb5_F | GGAATTCATGGATGGTCCCAATTTTGCAC | 27 |
| Stb5_R | ACGCGTCGACTCATACAAGTTTATCAACCCAAGA | 28 |
| | GACG | |

For the overexpression of the Gnd1 gene, Gdh2 gene, Ald6 gene, Zwf1 gene, or Stb5 gene, the p416_GPD_Gnd1 vector, p416_GPD_Gdh2 vector, p416_GPD_Ald6 vector, p416_GPD Zwf1 vector, or p416_GPD_Stb5 vector prepared above was transformed into each wild-type yeast strain of S. cerevisiae CEN.PK2-1D, respectively. The transformation was performed by conventional heat shock transformation, and the cells were cultured in a uracil drop-out medium so that only those strains where a vector containing a particular gene to be a subject for overexpression and URA3 were able to grow.

As a result, the prepared strains were named as CEN.PK2-1D+Gnd1, CEN.PK2-1D+Gdh2, CEN.PK2-1D+Ald6, CEN.PK2-1D+Zwf1, and CEN.PK2-1D+Stb5, respectively.

### 2-3: Preparation of Strains Where Ald2 is Inactivated and Ald6 is Overexpressed, and Strains where Gdh1 is Inactivated and Gdh2 is Overexpressed

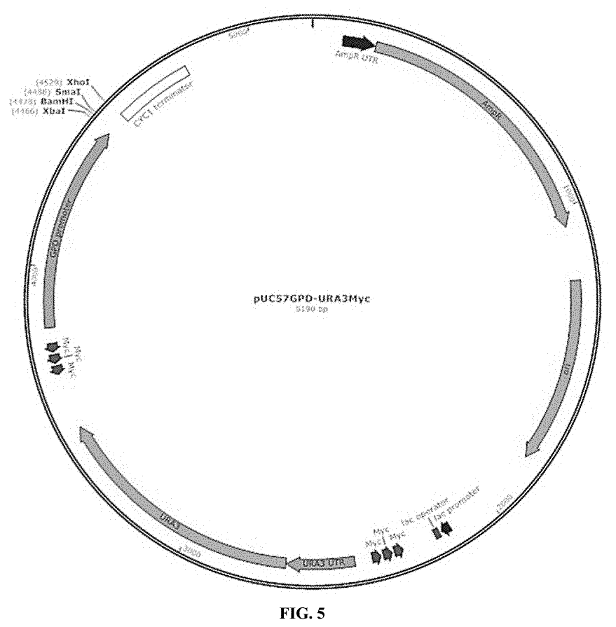

For the inactivation of the Ald2 gene and overexpression of the Ald6 gene, the Ald6 gene was amplified from the genomic DNA of S. cerevisiae CEN.PK2-1D by performing PCR using a primer set of Ald6 F and Ald6 R (Table 2) and the amplified products were digested with BamHI and XhoI, and ligated to the pUC57GPD-URA3Myc vector (Ju Young Lee et al., (2015) Biotechnol. Bioeng., 112, 751 to 758), which was also digested with BamHI and XhoI, and thereby the pUC57GPD-URA3Myc_Ald6 vector was prepared (FIG. 5 and SEQ ID NO: 30). Furthermore, for the inactivation of the Ald2 gene and overexpression of the Ald6 gene, a cassette that substitutes the Ald2 gene with the Ald6 gene was prepared by performing PCR using the above-prepared pUC57GPD-URA3Myc_Ald6 vector as a template and Del_Ald2_F and Del_Ald2_R, the homologous recombinant sequences of Ald2 gene region on the genome, as primers.

For the inactivation of the Gdh1 gene and overexpression of Gdh2 gene, the pUC57GPD-URA3Myc_Gdh2 vector was prepared in the same manner as in Example 2-2 by performing PCR using the primer set Gdh2_F and Gdh2_R (Table 2) and ligation by digestion with XbaI and SmaI. Furthermore, for the inactivation of the Gdh1 gene and overexpression of Gdh2 gene, a cassette that substitutes the Gdh1 gene with the Gdh2 gene was prepared by performing PCR using the above-prepared pUC57GPD-URA3Myc_Gdh2 vector as a template and Del_Gdh1_F and Del_Gdh1_R, the homologous recombinant sequences of Gdh1 gene region on the genome, as primers.

The prepared cassette was transformed into the cells of each wild-type yeast strain of S. cerevisiae CEN.PK2-1D. The transformation was performed by conventional heat shock transformation. After the transformation, the cells were cultured in a uracil drop-out medium so that the Ald2 gene or Gdh1 gene on the genome was able to be substituted with the cassette containing URA3.

The substitution of the Ald2 gene with Ald6 gene and the substitution of Gdh1 gene with Gdh2 gene in the strain were confirmed by performing PCR using the genomic DNA of the above cells as a template and a primer set of Ald2_conf_F and Ald2_conf_R and a primer set of Gdh1_conf_F and Gdh1_conf_R, respectively.

As a result, the prepared strains were named as CEN.PK2-1D,Δald2::ald6 and CEN.PK2-1D,ΔGdh1::Gdh2, respectively.

### EXAMPLE 3

### Confirmation of Amount of NADPH Production in Wild-Type Yeast and Transformed Yeast

A wild-type yeast strain, S. cerevisiae CEN.PK2-1D, and transformed yeast strains therefrom were inoculated into 50 mL of minimal uracil drop-out media containing 2% glucose such that the absorbance at OD₆₀₀ became 0.5, and cultured while stirring at 30° C. at a rate of 250 rpm under aerobic conditions for 24 hours.

At the time of terminating the culture, the amount of nicotinamide adenine dinucleotide phosphate (NAPDH) in the cell was analyzed using the EnzyChrom™ NADP⁺/NADPH assay kit (ECNP-100). The accompanying procedures of the analysis method were adjusted to be suitable for the experimental conditions as follows.

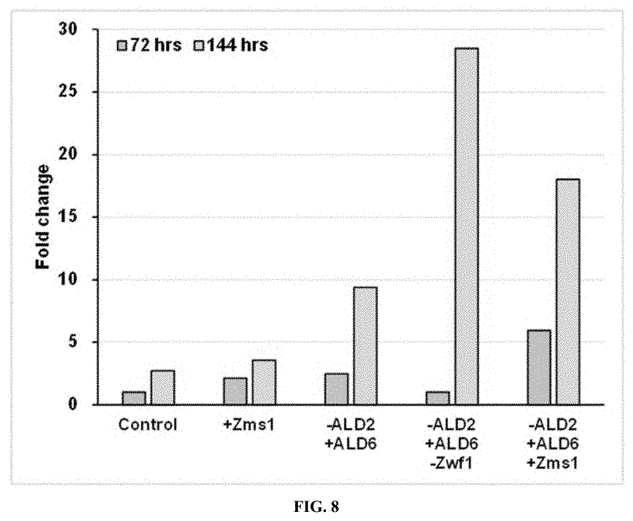

Specifically, the cell culture containing 10 OD (about 8×10⁹ cells) based on the OD₆₀₀ measurement using a spectrophotometer was separated by a centrifuge, the supernatant was discarded, and the cells in the form of a pellet were collected, washed with a cold PBS buffer, and mixed with an NADPH extraction buffer. The resultant was heated at 60° C. for 5 minutes, and an assay buffer (20 μL) and an NADPH extraction buffer (100 μL) were added thereto and mixed well. The mixed solution was centrifuged at 14,000 rpm for 5 minutes using a centrifuge, and the sample values set at 565 nm were measured using only the supernatant by a spectrophotometer. The measured values were substituted into a calibration curve and the concentration values of NADPH were obtained. The concentration values of NADPH after 24 hours of culture are shown in FIG. 8 below.

Specifically, as shown in FIG. 8, it was confirmed that the NADPH concentration in the cells of each of the strain with overexpression of Zwf1 gene (CEN.PK2-1D+Zwf1), the strain with overexpression of Stb5 gene (CEN.PK2-1D+STB5), the strain with inactivation of Ald2 gene and overexpression of Ald6 gene (CEN.PK2-1D, Δald2::ald6), and the strain with inactivation of Gdh1 gene and overexpression of Gdh2 gene (CEN.PK2-1D, ΔGdh1::Gdh2) were shown to be higher than that of the control group.

### EXAMPLE 4

### Confirmation of Growth and Amount of PPD Production of Transformed Modified Strains

The strains where the cellular concentration of NADH was increased compared to that of the control group were selected in Example 3, and the effects of the genes from these strains, which affect the NADPH biosynthetic pathway, on the growth of PPD yeast cells and their PPD production were examined.

### 4-1: Preparation of Strains Where Zwf1 or Stb5 is Overexpressed in PPD Yeast Cells

First, the Zwf1 gene and Stb5 gene were introduced into PPD yeast cells and overexpressed therein, and the effect of the overexpression of these genes on the growth of PPD yeast cells and their PPD production were examined.

Specifically, for overexpression of the Zwf1 gene or Stb5 gene in PPD yeast cells, and the p416_GPD_Zwf1 vector and the p416_GPD_Stb5 vector prepared in Example 1 were introduced into the PPD yeast strain, respectively. The transformation was performed by conventional heat shock transformation, and the cells were cultured in a uracil drop-out medium so that only those strains where the p416_GPD_Zwf1 vector or the p416_GPD_Stb5 vector containing URA3 was introduced were able to grow.

As a result, the prepared strains were named as PPD, p416_GPD_Zwf1 and PPD, p416_GPD_Stb5, respectively.

### 4-2: Preparation of Yeast Strains Where Ald2 is Inactivated and Ald6 is Overexpressed, and Strains Where Gdh1 is Inactivated and Gdh2 is Overexpressed in PPD Yeast Cells

In PPD yeast cells, the expression of the Ald2 gene was inhibited by substituting it with Ald6 gene while simultaneously overexpressing the Ald6 gene under the strong GPD promoter, and the effect of the overexpression of the Ald6 gene on the growth of PPD yeast cells and PPD production were examined. In the same manner, Gdh1 gene was inhibited by substituting it with Gdh2 gene and then the Gdh2 gene was overexpressed, and the effect of the overexpression of the Gdh2 gene on the growth of PPD yeast cells and PPD production were examined.

Specifically, for the inactivation of the Ald2 gene and overexpression ofAld6 gene, and the inactivation of the Gdh1 gene and overexpression of the Gdh2 gene in PPD yeast cells, a DNA cassette for homologous recombination was obtained in the same manner as described above, using pUC57GPD-URA3Myc_Ald6 and pUC57GPD-URA3Myc_Gdh2. The prepared cassette was introduced into each PPD yeast strain. The transformation was performed by conventional heat shock transformation. After the transformation, the cells were cultured in a uracil drop-out medium so that only the Ald2 gene or Gdh1 gene on the genome was able to be substituted with the cassette containing URA3. The substitution of the Ald2 gene with Ald6 gene and the substitution of Gdh1 gene with Gdh2 gene in the obtained strains was confirmed by performing PCR using the genomic DNA of the above cells as a template and a primer set of Ald2_conf_F and Ald2_conf_R and a primer set of Gdh1_conf_F and Gdh1_conf_R, respectively.

As a result, the prepared strains were named as PPD,Δald2::ald6 and PPD,ΔGdh1::Gdh2, respectively.

### 4-3: Preparation of Strains Where Zwf1 is Inactivated in PPD Yeast Cells

In the strains prepared in Example 4-2, the Zwf1 gene was further inactivated so as to control the PP pathway, and the effect of the detoured carbon-flux due to the inactivation of the Zwf1 gene on the growth of PPD yeast cells and their PPD production were examined.

Specifically, a cassette that inactivates the Zwf1 gene was obtained by performing PCR using the pUC57-URA3Myc vector (Ju Young Lee et al., (2015) Biotechnol. Bioeng., 112, 751 to 758) prepared above as a template and Del_Zwf1_F and Del_Zwf1_R, the homologous recombinant sequences of Zwf1 gene region on the genome, as primers. The prepared inactivation cassette was transformed into the yeast strain of PPD,Δald2::ald6. The transformation was performed by conventional heat shock transformation. After the transformation, the cells were cultured in a uracil drop-out medium so that the Zwf1 gene on the genome was able to be substituted with the cassette containing URA3.

The inactivity of the Zwf1 gene in the obtained strain was confirmed by performing PCR using the genomic DNA of the above cells as a template and a primer set of Zwf1_conf_F and Zwf1_conf_R, and the strain was named as PPD,Δald2::a1d6,Δzwf1. The primers used above are shown in Table 3 below.

TABLE 3

| | | SEQ |
|---|---|---|
| | | ID |
| Name | Primer Sequence (5′→3′) | NO |
| Del_Zwfl_F | TATAGACAGAAAGAGTAAATCCAATAGAAT | 34 |
| | AGAAAACCACATAAGGCAAGCCAGTCACGA | |
| | CGTTGTAAAA | |
| Del_Zwfl_R | CCTCCCAACGCTCGTTTTCGATGTTGAAAG | 35 |
| | TCATTGCTGCAAAAGTGACAAGGTTTCCCG | |
| | ACTGGAAAGC | |
| Zwfl_conf_F | AGAATAGAAAACCACATAAGGCAAG | 36 |
| Zwfl_conf_R | CCTCCCAACGCTCGTTTTCG | 37 |

### 4-4: Preparation of Vector for Overexpression of Zms1 in PPD Yeast Cells

Additionally, in the strains prepared in Example 4-2, Zms1 gene was overexpressed and the effects of the overexpression of the Zms1 gene on the growth of PPD yeast cells and PPD production were examined.

Specifically, for the overexpression of the Zms1 gene, the Zms1 gene was amplified by performing PCR using the genomic DNA of S. cerevisiae CEN.PK2-1D and a primer set of Zms1_F and Zms1_R. The amplified PCR products were digested with XmaI and XhoI and then ligated to the p416_GPD vector, which was also digested with XmaI and XhoI, and thereby the p416_GPD_Zms1 vector was prepared. The primers used above are shown in Table 4 below.

TABLE 4

| | | SEQ |
|---|---|---|
| | | ID |
| Name | Primer Sequence (5′→3′) | NO |
| Zms1_F | TAGTGGATCCCCCGGGATGTTTGTGAACGGT | 38 |
| | AATCAATCTAATTTC | |
| Zms1_R | AATTACATGACTCGAGTTATATTCTAGTGTT | 39 |
| | TCTTTTTTTCGTAAC | |

For the overexpression of the Zms1 gene, the p416_GPD_Zms1 vector prepared above was introduced into each of the PPD yeast strain and the PPD,Δald2::ald6 strain. The transformation was performed by conventional heat shock transformation, and the cells were cultured in a uracil drop-out medium so that only those strains where the p416_GPD_Zms1 vector containing URA3 introduced were able to grow.

As a result, the prepared strains were named as PPD,p416_GPD_Zms1 and PPD,Δald2::ald6,p416_GPD_Zms1, respectively.

### EXAMPLE 5

### Confirmation of Growth of Transformed Yeast Strains and Their PPD Production

The transformed yeast strains prepared above were inoculated into 50 mL of minimal URA drop-out media containing 2% glucose such that the absorbance at OD₆₀₀ became 0.5, and cultured while stirring at 30° C. at a rate of 250 rpm under aerobic conditions for 144 hours. The OD₆₀₀ values of the cells during the culture were measured using a spectrophotometer. The intracellular metabolites (i.e., squalene, 2,3-oxidosqualene, and protopanaxadiol) were analyzed by high performance liquid chromatography (HPLC).

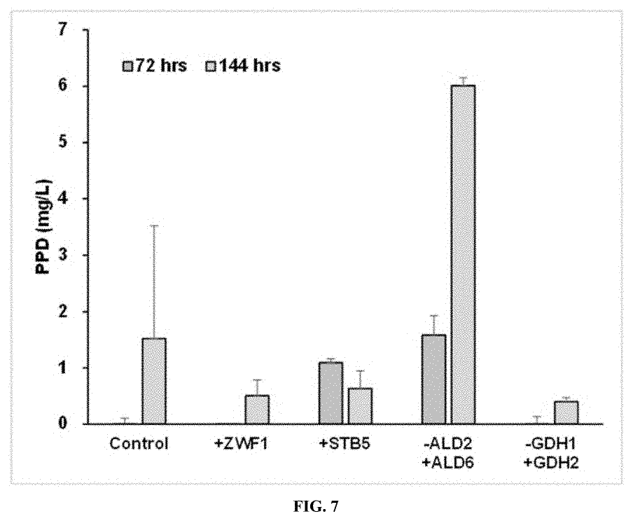

As a result, the cell growth (i.e., the OD₆₀₀ values of the cell culture) and the concentration of each intracellular metabolite are shown in the following Table 5, and FIGS. 7 and 8.

TABLE 5
Concentration of metabolites according to cultivation
of transformed modified yeast strains

| | |
|---|---|
| OD₆₀₀ | Protopanaxadiol (mg/L) |

| | 72 h | 144 h | 72 h | 144 h |
|---|---|---|---|---|
| Control | 26.75 | 24.62 | 0 | 1.52 |
| +Zwf1 | 26.03 | 22.91 | 0.03 | 0.51 |
| +STB5 | 19.70 | 20.80 | 1.09 | 0.64 |
| −Ald2 + Ald6 | 13.19 | 11.88 | 1.58 | 6.01 |
| −Gdh1 + Gdh2 | 24.82 | 24.08 | 0 | 0.40 |

Specifically, referring to the results of FIG. 7, it was confirmed that the amounts of PPD production in the strains where the Ald2 gene was inactivated and the Ald6 gene was overexpressed, after 4 hours of culture, were about 4-fold greater compared to that of the control group. Additionally, it was confirmed that in the strains where the Zwf1 gene, which affects the NADPH biosynthetic pathway, was further inactivated or where the Zms1 gene was further overexpressed, the amount of protopanaxadiol production was significantly increased, and in particular, in the strains where the Zwf1 gene was further inactivated, the amount of protopanaxadiol production was about 20-fold or greater compared to that of the control group (FIG. 8).

From the above results, it was confirmed that when the expression levels of NADPH biosynthesis-related proteins (Ald2, Ald6, Zwf1, and Zms1) were changed compared to their endogenous expression levels, the amount of the protopanaxadiol production was significantly increased.

From the foregoing, a skilled person in the art to which the present invention pertains will be able to understand that the present invention may be embodied in other specific forms without modifying the technical concepts or essential characteristics of the present invention. In this regard, the exemplary embodiments disclosed herein are only for illustrative purposes and should not be construed as limiting the scope of the present invention. On the contrary, the present invention is intended to cover not only the exemplary embodiments but also various alternatives, modifications, equivalents, and other embodiments that may be included within the spirit and scope of the present invention as defined by the appended claims.

1. Yeast for producing ginsenosides, wherein the expression levels of NADPH biosynthesis-related genes are changed compared to their endogenous expression levels.

2. The yeast of claim 1, wherein the NADPH biosynthesis-related genes are one or more genes selected from the group consisting of Ald2, Ald6, Zwf1, and Zms1.

3. The yeast of claim 2, wherein the expression level of Ald2 is decreased compared to its endogenous expression level and the expression level of Ald6 is increased compared to its endogenous expression level.

4. The yeast of claim 3, wherein the expression level of Zwf1 is further decreased compared to its endogenous expression level.

5. The yeast of claim 3, wherein the expression level of Zms1 is further increased compared to its endogenous expression level.

6. The yeast of claim 1, wherein the NADPH-producing ability is increased compared to its endogenous level.

7. The yeast of claim 1, wherein the expression levels of ginsenoside synthesis-related genes are further increased compared to their endogenous expression levels.

8. The yeast of claim 7, wherein the genes are one or more selected from the group consisting of PgDDS (Panax ginseng, dammarenediol-II synthase), PgPPDS (Panax ginseng cytochrome P450 CYP716A47), PgCPR (Panax ginseng, NADPH-cytochrome P450 reductase), tHMG1 (S. cerevisiae HMG-CoA reductase), and PgSE (Panax ginseng, squalene epoxidase).

9. A method for preparing recombinant yeast with an enhanced ginsenoside-producing ability, comprising changing the expression levels of the NADPH biosynthesis-related genes compared to their endogenous expression levels.

10. The method of claim 9, wherein the NADPH biosynthesis-related genes are one or more genes selected from the group consisting of Ald2, Ald6, Zwf1, and Zms1.

11. The method of claim 9, wherein the expression level of Ald2 is decreased compared to its endogenous expression level and the expression level of Ald6 is increased compared to its endogenous expression level.

12. The method of claim 11, wherein the expression level of Zwf1 is further decreased compared to its endogenous expression level.

13. The method of claim 11, wherein the expression level of Zms1 is further increased compared to its endogenous expression level.

14. The method of claim 9, wherein the expression levels of one or more genes selected from the group consisting of PgDDS (Panax ginseng, dammarenediol-II synthase), PgPPDS (Panax ginseng cytochrome P450 CYP716A47), PgCPR (Panax ginseng, NADPH-cytochrome P450 reductase), tHMG1 (S. cerevisiae HMG-CoA reductase), and PgSE (Panax ginseng, squalene epoxidase) are increased compared to their endogenous expression levels.

15. A method for producing ginsenosides, comprising:
(a) culturing the yeast of claim 1 in a medium; and
(b) recovering ginsenosides from the yeast or the medium.

16. A method for producing ginsenosides, comprising:
(a) culturing the yeast of claim 2 in a medium; and
(b) recovering ginsenosides from the yeast or the medium.


SEQUENCE LISTING (34 sequences)

SEQ ID NO: 1 - DNA, 1521 bp, Unknown
  Ald2
atgcctaccttgtatactgatatcgaaatcccacaattgaaaatctctttaaagcaaccgctagggttgtttatcaacaatgagttttgtccatcatcagatggaaagaccatcgaaactgtgaacccagctactggcgaaccgataacatccttccaagcagctaacgaaaaggatgtagacaaagctgtgaaagctgccagggctgcttttgataacgtttggtcgaagacatcttctgagcaacgtggtatttatctttcaaacttattaaaacttattgaggaggagcaagacacacttgccgcattagagactttagacgctggaaagccttaccattcaaatgccaaaggtgatttggcacaaattttacagcttaccagatattttgctgggtccgctgataagtttgacaaaggtgcaaccataccattgacttttaacaagtttgcatatactctaaaagttccttttggcgttgttgctcaaatcgttccatggaattatcctctagctatggcttgttggaaattgcaaggtgccttagcagccggtaacacggttatcatcaaacctgctgagaatacctctctatctctactttattttgctactttaattaaaaaagcaggttttccacctggtgttgtcaatatcgttcctggttatggatcacttgtaggccaagccctagcatctcacatggatatcgacaaaatatcttttacgggaagcaccaaggtcggtggatttgtgttggaagcttccggccaatcgaaccttaaagacgttacactagaatgcggtggtaagtctcctgctctcgtatttgaagatgcagaccttgataaggctatcgattggatagcagctggcattttctacaattcaggacagaattgtaccgcaaactcaagagtttatgttcaaagttcgatctacgacaagtttgttgaaaagtttaaagaaactgcaaagaaggagtgggatgttgcaggaaaatttgatccgtttgatgagaaatgcatcgttggtccagttatatcaagtacacagtatgaccgcatcaaaagttacatagaacgtggtaaaagggaggaaaagttggacatgttccagacctctgaatttcctattggtggagctaaaggctacttcattcccccaaccatcttcactgatgtcccgcaaacatcgaaactgttacaggatgagatatttggcccggttgtggttgttagcaagttcacaaattatgatgacgctctgaagctggctaatgatacttgctacgggctcgcctctgcggtcttcacaaaagatgtcaagaaagcgcacatgtttgctcgcgatattaaagcaggaactgtttggatcaactcatctaacgatgaagatgttaccgttccttttggcgggtttaaaatgagtggtattggtagagaactggggcaaagtggtgttgatacctatcttcaaacaaaagcagttcacataaatctctctttggacaactaa

SEQ ID NO: 2 - DNA, 1503 bp, Unknown
  Ald6
atgactaagctacactttgacactgctgaaccagtcaagatcacacttccaaatggtttgacatacgagcaaccaaccggtctattcattaacaacaagtttatgaaagctcaagacggtaagacctatcccgtcgaagatccttccactgaaaacaccgtttgtgaggtctcttctgccaccactgaagatgttgaatatgctatcgaatgtgccgaccgtgctttccacgacactgaatgggctacccaagacccaagagaaagaggccgtctactaagtaagttggctgacgaattggaaagccaaattgacttggtttcttccattgaagctttggacaatggtaaaactttggccttagcccgtggggatgttaccattgcaatcaactgtctaagagatgctgctgcctatgccgacaaagtcaacggtagaacaatcaacaccggtgacggctacatgaacttcaccaccttagagccaatcggtgtctgtggtcaaattattccatggaactttccaataatgatgttggcttggaagatcgccccagcattggccatgggtaacgtctgtatcttgaaacccgctgctgtcacacctttaaatgccctatactttgcttctttatgtaagaaggttggtattccagctggtgtcgtcaacatcgttccaggtcctggtagaactgttggtgctgctttgaccaacgacccaagaatcagaaagctggcttttaccggttctacagaagtcggtaagagtgttgctgtcgactcttctgaatctaacttgaagaaaatcactttggaactaggtggtaagtccgcccatttggtctttgacgatgctaacattaagaagactttaccaaatctagtaaacggtattttcaagaacgctggtcaaatttgttcctctggttctagaatttacgttcaagaaggtatttacgacgaactattggctgctttcaaggcttacttggaaaccgaaatcaaagttggtaatccatttgacaaggctaacttccaaggtgctatcactaaccgtcaacaattcgacacaattatgaactacatcgatatcggtaagaaagaaggcgccaagatcttaactggtggcgaaaaagttggtgacaagggttacttcatcagaccaaccgttttctacgatgttaatgaagacatgagaattgttaaggaagaaatttttggaccagttgtcactgtcgcaaagttcaagactttagaagaaggtgtcgaaatggctaacagctctgaattcggtctaggttctggtatcgaaacagaatctttgagcacaggtttgaaggtggccaagatgttgaaggccggtaccgtctggatcaacacatacaacgattttgactccagagttccattcggtggtgttaagcaatctggttacggtagagaaatgggtgaagaagtctaccatgcatacactgaagtaaaagctgtcagaattaagttgtaa

SEQ ID NO: 3 - DNA, 1518 bp, Unknown
  Zwf1
atgagtgaaggccccgtcaaattcgaaaaaaataccgtcatatctgtctttggtgcgtcaggtgatctggcaaagaagaagacttttcccgccttatttgggcttttcagagaaggttaccttgatccatctaccaagatcttcggttatgcccggtccaaattgtccatggaggaggacctgaagtcccgtgtcctaccccacttgaaaaaacctcacggtgaagccgatgactctaaggtcgaacagttcttcaagatggtcagctacatttcgggaaattacgacacagatgaaggcttcgacgaattaagaacgcagatcgagaaattcgagaaaagtgccaacgtcgatgtcccacaccgtctcttctatctggccttgccgccaagcgtttttttgacggtggccaagcagatcaagagtcgtgtgtacgcagagaatggcatcacccgtgtaatcgtagagaaacctttcggccacgacctggcctctgccagggagctgcaaaaaaacctggggcccctctttaaagaagaagagttgtacagaattgaccattacttgggtaaagagttggtcaagaatcttttagtcttgaggttcggtaaccagtttttgaatgcctcgtggaatagagacaacattcaaagcgttcagatttcgtttaaagagaggttcggcaccgaaggccgtggcggctatttcgactctataggcataatcagagacgtgatgcagaaccatctgttacaaatcatgactctcttgactatggaaagaccggtgtcttttgacccggaatctattcgtgacgaaaaggttaaggttctaaaggccgtggcccccatcgacacggacgacgtcctcttgggccagtacggtaaatctgaggacgggtctaagcccgcctacgtggatgatgacactgtagacaaggactctaaatgtgtcacttttgcagcaatgactttcaacatcgaaaacgagcgttgggagggcgtccccatcatgatgcgtgccggtaaggctttgaatgagtccaaggtggagatcagactgcagtacaaagcggtcgcatcgggtgtcttcaaagacattccaaataacgaactggtcatcagagtgcagcccgatgccgctgtgtacctaaagtttaatgctaagacccctggtctgtcaaatgctacccaagtcacagatctgaatctaacttacgcaagcaggtaccaagacttttggattccagaggcttacgaggtgttgataagagacgccctactgggtgaccattccaactttgtcagagatgacgaattggatatcagttggggcatattcaccccattactgaagcacatagagcgtccggacggtccaacaccggaaatttacccctacggatcaagaggtccaaagggattgaaggaatatatgcaaaaacacaagtatgttatgcccgaaaagcacccttacgcttggcccgtgactaagccagaagatacgaaggataattag

SEQ ID NO: 4 - DNA, 3798 bp, Unknown
  Zms1
atgtttgtgaacggtaatcaatctaatttcgctaagcccgctggtcaaggtattctgcccattcctaaaaaatctcgaattattaagactgataagccaagaccgttcttgtgtcccacatgcactaggggttttgtcaggcaggagcatttgaagagacatcagcattcgcatacccgtgagaaaccgtatctttgtatcttttgcggtaggtgttttgctcgtagagatttagtgctcaggcatcagcaaaaacttcatgctgctcttgtaggtacgggggatccacggcgaatgacgccagcaccaaattcgacttcttcttttgcctccaagcggcgccattccgtggcggcggatgatccaaccgaccttcatatcattaaaatagccggaaataaagagactattctacccaccccgaagaaccttgctggtaagacatctgaagaattgaaagaggccgtggttgccttggccaaatcaaataatgtagaacttcccgtctcggccccagtaatgaacgataagcgagagaaaactcctcctagtaaggcaggctccctaggatttcgagagttcaagttcagcacgaaaggcgtgccagttcactctgcatcaagcgatgctgttatcgacagggcgaacactccctcttccatgcataagacgaaaagacatgcgtctttctctgcatccagtgcaatgacttacatgtctagtagcaatagcccccaccattcaattaccaatttcgagctcgttgaagacgctccgcatcaagtcggcttttctactccacaaatgaccgcgaagcagctcatggaaagcgtgtcagaattggatttacctccgttaaccctggacgaaccaccgcaagctatcaagtttaacttaaatctatttaacaatgacccctccggacagcaacaacaacaacaacaacaacagcaaaattccacctctagtaccatagtgaacagcaacaatggaagtacagttgctacacctggagtgtatctcttaagtagcggtccatctttaaccgatcttttgacaatgaactctgcacatgcaggtgcgggaggatacatgtctagccaccattcgccatttgatttgggctgcttcagtcatgataaaccgacagtttctgaatttaaccttccgtcaagcttcccgaatactataccgtctaattctactacggcttctaatagttacagtaatttggcaaatcaaacttataggcaaatgagcaatgagcagccgcttatgtcactatctcctaaaaacccaccaacaactgtttcagattcctcttccacgatcaatttcaatccaggcacaaataatttactggaaccatcaatggagcccaatgataaggatagtaatatcgatcctgctgccatagatgacaagtggttatcagagtttattaacaactctgatccaaaatctaccttcaagatcaacttcaatcatttcaatgacattgggtttatttattctccaccttcatcaaggtcatctataccaaacaagtcacctccaaaccattctgctacctcattaaatcatgaaaaagcttctttatcacctcgcttaaacttgagtttgaatggaagcacagatttaccaagtacaccacaaaaccaactaaaggagccttcctattctgaccctatttcccatagttctcataagaggcgtcgtgatagcgtcatgatggactacgatctatccaattttttcagctcaaggcaattggatatttccaaggtattaaacgggacagagcaaaataattctcatgtgaacgacgatgttctcactttgtctttccccggcgaaactgattctaatgcaacacagaaacagctgcctgttcttactccttcggatttgttatctccgttttctgtcccttcagtatctcaagtgctttttaccaatgagctaaggagtatgatgctagccgacaataatatcgattcaggagccttccccacaactagtcaattgaacgattatgtgacttactataaggaagaattccatccatttttttcatttattcatcttccttctatcatacctaatatggacagttatcccttgttattatctatctccatggtcggagcattgtatgggtttcattcgacgcatgcaaaagtgttagctaatgcagctagcacccaaattaggaaaagcttgaaagttagtgagaaaaacccggagacgacagagttatgggttatacagacattagtattgctaacgttctactgtattttcaataaaaatacagccgtgatcaaggggatgcatggtcagttgacgactattattcgtctcttgaaggcctctcgtttaaatttgcccctagagtccctatgccagccgcctattgagagtgatcatattatggaatatgaaaacagtcctcatatgttttcaaaaataagagagcaatacaacgcgccgaatcaaatgaacaaaaactaccaatattttgtattggcgcagtcacgtatcaggacttgccatgcggtattacttatatctaacttattttcttcactggtaggtgctgattgctgttttcattcagtcgatttaaaatgtggtgttccatgctataaagaagaattatatcagtgccgaaattccgatgaatggtcggacctattatgtcaatacaaaataacgttagattcgaaattttcgttgattgaattgtctaatggtaacgaggcatatgaaaattgtttgaggtttctttctacaggcgatagttttttttacggaaatgctagggtttcgttaagtacatgtctatcattgttgatatctatccatgagaaaatacttattgaaagaaataacgcaaggatcagtaataacaacaccaatagcaataacattgagttggacgatattgagtggaagatgacttccagacaacggatcgatacaatgttaaaatactgggaaaacctttatttgaaaaatggtggcatcttgacacctaccgagaatagcatgtcaacaataaacgccaatccagcaatgaggttaataattccggtatatttgtttgccaaaatgagacggtgtttggacctggcacatgttattgagaaaatctggttgaaagattggtccaatatgaataaagctttggaggaagtttgctatgacatgggttcattgagggaagctaccgagtatgcactgaatatggtggatgcgtggacttcattttttacgtacattaaacagggcaagcgcagaattttcaatactcctgtatttgcgaccacatgtatgttcactgcagtattagtgatttcggaatacatgaaatgtgtagaggattgggcacgcgggtacaatgccaacaaccctaactcagcattattggatttttcggaccgtgtcttatggctaaaagcagaaaggattttgagaagattacaaatgaacttgataccgaaggagtgtgatgtgttgaaatcgtacactgatttcttaagatggcaggacaaggatgccctagatttgtcagcactaaatgaagaacaagcacaaagggccatggacccgaataccgatataaatgagacaattcaactaattgtagcggcaagtctatcctccaaatgtttatatttgggtgttcaaatattgggtgatgcgccaatttggcctataatattatcgttcgctcatggtttgcaatcaagagctatctatagtgttacgaaaaaaagaaacactagaatataa

SEQ ID NO: 5 - protein, 527 aa, Unknown
  tHMG1(S. cerevisiae HMG-CoA reductase)
MAADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 6 - protein, 545 aa, Unknown
  PgSE (Panax ginseng, squalene epoxidase)
MELERSYRENDEYFLMFAATLLFGFVLYLFTLRRRRRRREKKGGAGSMEIINGAYKMTSSSEVNGHCTPEDIAGSSDDVIIVGAGVAGSALAYTLAKDGRRVHVIERDLTEQDRIVGELLQPGGYLKLVELGLEDCVNEIDAQRVFGYALYMDGKNTRLSYPLEKFHADVAGRSFHNGRFIQRMREKAASLPNVRMEQGTVTSLVEQKGTVKGVRYKTKNGQEMSAAYAPLTIVCDGCFSNLRHSLCNPKVDVPSCFVGLILENIDLPHINHGHVILADPSPILFYKISSTEIRCLVDVPGQKVPSIANGELAHYLKTSVAPQIPPELYKSFIAAIDKGKIKTMPNRSMPADPHSTPGALLLGDAFNMRHPLTGGGMTVALSDIVLIRDLLRPLRDLHDSSTLCKYLESFYTLRKPVASTINTLAGALYKVFCASPDKARQEMRDACFDYLSLGGICSEGPIALLSGLNPRPMSLFFHFFAVAIYGVGRLLIPFPSPRKMWLGARLISGASGIIFPIIKSEGVRQMFFPATVPAYYRAPPITKKM

SEQ ID NO: 7 - protein, 769 aa, Unknown
  PgDS (Panax ginseng, dammarenediol-II synthase)
MWKQKGAQGNDPYLYSTNNFVGRQYWEFQPDAGTPEEREEVEKARKDYVNNKKLHGIHPCSDMLMRRQLIKESGIDLLSIPPLRLDENEQVNYDAVTTAVKKALRLNRAIQAHDGHWPAENAGSLLYTPPLIIALYISGTIDTILTKQHKKELIRFVYNHQNEDGGWGSYIEGHSTMIGSVLSYVMLRLLGEGLAESDDGNGAVERGRKWILDHGGAAGIPSWGKTYLAVLGVYEWEGCNPLPPEFWLFPSSFPFHPAKMWIYCRCTYMPMSYLYGKRYHGPITDLVLSLRQEIYNIPYEQIKWNQQRHNCCKEDLYYPHTLVQDLVWDGLHYFSEPFLKRWPFNKLRKRGLKRVVELMRYGATETRFITTGNGEKALQIMSWWAEDPNGDEFKHHLARIPDFLWIAEDGMTVQSFGSQLWDCILATQAIIATNMVEEYGDSLKKAHFFIKESQIKENPRGDFLKMCRQFTKGAWTFSDQDHGCVVSDCTAEALKCLLLLSQMPQDIVGEKPEVERLYEAVNVLLYLQSRVSGGFAVWEPPVPKPYLEMLNPSEIFADIVVEREHIECTASVIKGLMAFKCLHPGHRQKEIEDSVAKAIRYLERNQMPDGSWYGFWGICFLYGTFFTLSGFASAGRTYDNSEAVRKGVKFFLSTQNEEGGWGESLESCPSEKFTPLKGNRTNLVQTSWAMLGLMFGGQAERDPTPLHRAAKLLINAQMDNGDFPQQEITGVYCKNSMLHYAEYRNIFPLWALGEYRKRVWLPKHQQLKI

SEQ ID NO: 8 - protein, 482 aa, Unknown
  PgPPDS (Panax ginseng cytochrome P450 CYP716A47)
MVLFFSLSLLLLPLLLLFAYFSYTKRIPQKENDSKAPLPPGQTGWPLIGETLNYLSCVKSGVSENFVKYRKEKYSPKVFRTSLLGEPMAILCGPEGNKFLYSTEKKLVQVWFPSSVEKMFPRSHGESNADNFSKVRGKMMFLLKVDGMKKYVGLMDRVMKQFLETDWNRQQQINVHNTVKKYTVTMSCRVFMSIDDEEQVTRLGSSIQNIEAGLLAVPINIPGTAMNRAIKTVKLLTREVEAVIKQRKVDLLENKQASQPQDLLSHLLLTANQDGQFLSESDIASHLIGLMQGGYTTLNGTITFVLNYLAEFPDVYNQVLKEQVEIANSKHPKELLNWEDLRKMKYSWNVAQEVLRIIPPGVGTFREAITDFTYAGYLIPKGWKMHLIPHDTHKNPTYFPSPEKFDPTRFEGNGPAPYTFTPFGGGPRMCPGIEYARLVILIFMHNVVTNFRWEKLIPNEKILTDPIPRFAHGLPIHLHPHN

SEQ ID NO: 9 - protein, 709 aa, Unknown
  PgCPR (Panax NADPH-cytochrome P450 reductase)
MLKVSPFDLMTEILRGGSIDPPNSSVSAAGASMQPSLAMLVVNRELLMLLTTSVAVLIGCVVVLVWRKSSSQKHAKSFEAPKLLIPKIEPEEVVDDGKKKVTIFFGTQTGTAEGFAKALAEEAKARYEKAIFKVIDLDDYAPEDDDYETKLKKESLAFFFLATYGDGEPTDNAARFYKWFTEGKEKREWLNNLQYGVFGLGNRQYEHFNKIAKVVDDGLAEQGAKRLVPVGMGDDDQCIEDDFTAWRELVWPELDQLLLDEEDTAAATPYTAAVLEYRVVFHDRTDSSTLLNGTTSVSNGHAFYDAQHPCRANVAVKRELHTLESDRSCTHLEFDISSTGLAYETGDHVGVYTENLIEIVEEAERLLAISPDTYFSIHTEKEDGSPVSGSSLQPPFPPCTLREALRRYADLLSSPKKSALLALAAHASDPSEADRLRFLASPAGKDEYAQWIVANQRSLLEVLAEFPSAKPPLGVFFASVAPRLQPRYYSISSSPRMAPSRIHVTCALVFERTPAGRIHKGVCSTWMKNAVSLEEGNDCSRAPIFVRQSNFKLPSDSRMPIIMIGPGTGLAPFRGFLQERLALKEAGAELGPAVLYFGCRNRKLDFIYEDELNNFVESGAISEMVVAFSREGPTKEYVQHKMSQKASEIWNMISEGAYIYVCGDAKGMARDVHRTLHTIAQEQGALDSSKAESLVKNLQMTGRYLRDVW

SEQ ID NO: 10 - DNA, 70 bp, Artificial Sequence
  Del_Ald2_F
ttacattgcatgtccatcaaaaacaatcgtgaaaataagccaaaagaaaaccagtcacgacgttgtaaaa

SEQ ID NO: 11 - DNA, 70 bp, Artificial Sequence
  Del_Ald2_R
ctgcaacatcccactccttctttgcagtttctttaaacttttcaacaaacaggtttcccgactggaaagc

SEQ ID NO: 12 - DNA, 25 bp, Artificial Sequence
  Ald2_conf_F
ttacattgcatgtccatcaaaaaca

SEQ ID NO: 13 - DNA, 20 bp, Artificial Sequence
  Ald2_conf_R
ctgcaacatcccactccttc

SEQ ID NO: 14 - DNA, 70 bp, Artificial Sequence
  Del_Gdh1_F
actatcgcattattctaatataacagttaggagaccaaaaagaaaaagaaccagtcacgacgttgtaaaa

SEQ ID NO: 15 - DNA, 70 bp, Artificial Sequence
  Del_Gdh1_R
gacggcaatagcttctggagtggaacccatgttggaaccttcggcaataaaggtttcccgactggaaagc

SEQ ID NO: 16 - DNA, 27 bp, Artificial Sequence
  Gdh1_conf_F
cagttaggagaccaaaaagaaaaagaa

SEQ ID NO: 17 - DNA, 20 bp, Artificial Sequence
  Gdh1_conf_R
gacggcaatagcttctggag

SEQ ID NO: 18 - DNA, 4226 bp, Unknown
  pUC57-URA3Myc
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcccgacctggagctgtgcggccgttctgagcaaaagctcatttctgaagaggacttgaatggagaacagaaattgatcagtgaggaagacctcaacggtgagcagaagttaatatccgaggaggatcttaatagtagcggccgtactagcggatttccccgcggccgctacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaaggtggtatggtgcactctcaggcggccgcgaattagagctgtgcggccgttctgagcaaaagctcatttctgaagaggacttgaatggagaacagaaattgatcagtgaggaagacctcaacggtgagcagaagttaatatccgaggaggatcttaatagtagcggccgtactagcggggggcccggagacgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 19 - DNA, 27 bp, Artificial Sequence
  Gnd1_F
ggaattcatgtctgctgatttcggttt

SEQ ID NO: 20 - DNA, 34 bp, Artificial Sequence
  Gnd1_R
ccgctcgagttaagcttggtatgtagaggaagaa

SEQ ID NO: 21 - DNA, 34 bp, Artificial Sequence
  Gdh2_F
gctctagaatgctttttgataacaaaaatcgcgg

SEQ ID NO: 22 - DNA, 29 bp, Artificial Sequence
  Gdh2_R
tcccccgggtcaagcacttgcctccgctt

SEQ ID NO: 23 - DNA, 34 bp, Artificial Sequence
  Ald6_F
cgggatccatgactaagctacactttgacactgc

SEQ ID NO: 24 - DNA, 41 bp, Artificial Sequence
  Ald6_R
ccgctcgagttacaacttaattctgacagcttttacttcag

SEQ ID NO: 25 - DNA, 27 bp, Artificial Sequence
  Zwf1_F
ggaattcatgagtgaaggccccgtcaa

SEQ ID NO: 26 - DNA, 34 bp, Artificial Sequence
  Zwf1_R
ccgctcgagctaattatccttcgtatcttctggc

SEQ ID NO: 27 - DNA, 29 bp, Artificial Sequence
  Stb5_F
ggaattcatggatggtcccaattttgcac

SEQ ID NO: 28 - DNA, 38 bp, Artificial Sequence
  Stb5_R
acgcgtcgactcatacaagtttatcaacccaagagacg

SEQ ID NO: 29 - DNA, 5778 bp, Unknown
  p416_GPD vector
gacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctcagtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaactagtggatcccccgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 30 - DNA, 5190 bp, Unknown
  pUC57GPD-URA3Myc
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaggcctctgcagtcgacgggcccgtctccgggccccccgctagtacggccgctactattaagatcctcctcggatattaacttctgctcaccgttgaggtcttcctcactgatcaatttctgttctccattcaagtcctcttcagaaatgagcttttgctcagaacggccgcacagctctaattcgcggccgcctgagagtgcaccataccaccttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtagcggccgcggggaaatccgctagtacggccgctactattaagatcctcctcggatattaacttctgctcaccgttgaggtcttcctcactgatcaatttctgttctccattcaagtcctcttcagaaatgagcttttgctcagaacggccgcacagctccaggtcgggatcggaacaaaagctggagctcagtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaactagtggatcccccgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtaaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 31 - DNA, 1365 bp, Unknown
  Gdh1
atgtcagagccagaatttcaacaagcttacgaagaagttgtctcctctttggaagactctactcttttcgaacaacacccagaatacagaaaggttttgccaattgtttctgttccagaaagaatcatacaattcagagtcacctgggaaaatgacaagggtgaacaagaagttgctcaaggttacagagtgcaatataactccgccaagggtccatacaagggtggtctacgtttccatccttccgtgaacttgtctatcttgaaattcttgggtttcgaacaaatcttcaagaactccttgaccggcctagacatgggtggtggtaaaggtggtctatgtgtggacttgaagggaagatctaataacgaaatcagaagaatctgttatgctttcatgagagaattgagcagacacattggtcaagacactgacgtgccagctggtgatatcggtgttggtggtcgtgaaattggttacctgttcggtgcttacagatcatacaagaactcctgggaaggtgtcttaaccggtaagggtttgaactggggtggttctttgatcagaccagaagccactggttacggtttagtttactatactcaagctatgatcgactatgccacaaacggtaaggaatctttcgaaggtaagcgcgtcaccatctctggtagtggtaacgttgctcaatacgctgccttgaaggttattgagctaggtggtactgtcgtttccctatctgactccaagggttgtatcatctctgaaactggtatcacctccgaacaagtcgctgatatttccagtgctaaggtcaacttcaagtccttggaacaaatcgtcaacgaatactctactttctccgaaaacaaagtgcaatacattgctggtgctcgtccatggacccacgtccaaaaggtcgacattgctttgccatgtgccacccaaaatgaagtcagcggtgaagaagccaaggccttggttgctcaaggtgtcaagtttattgccgaaggttccaacatgggttccactccagaagctattgccgtctttgaaactgctcgttccaccgccactggaccaagcgaagctgtttggtacggtccaccaaaggctgctaacttgggtggtgttgctgtttctggtttagaaatggcacaaaactctcaaagaatcacatggactagcgaaagagttgaccaagagttgaagagaattatgatcaactgtttcaatgaatgtatcgactatgccaagaagtacactaaggacggtaaggtcttgccatctttggtcaaaggtgctaatatcgcaagtttcatcaaggtctctgatgctatgtttgaccaaggtgatgtattttaa

SEQ ID NO: 32 - DNA, 1470 bp, Unknown
  Gnd1
atgtctgctgatttcggtttgattggtttggccgtcatgggtcaaaatttgatcttgaacgctgctgaccacggtttcactgtttgtgcttacaacagaactcaatccaaggtcgaccatttcttggccaatgaagctaagggcaaatctatcatcggtgctacttccattgaagatttcatctccaaattgaagagacctagaaaggtcatgcttttggttaaagctggtgctccagttgacgctttgatcaaccaaatcgtcccacttttggaaaagggtgatattatcatcgatggtggtaactctcacttcccagattctaatagacgttacgaagaattgaagaagaagggtattcttttcgttggttctggtgtctccggtggtgaggaaggtgcccgttacggtccatctttgatgccaggtggttctgaagaagcttggccacatattaagaacatcttccaatccatctctgctaaatccgacggtgaaccatgttgcgaatgggttggcccagccggtgctggtcactacgtcaagatggttcacaacggtattgaatacggtgatatgcaattgatttgtgaagcttatgacatcatgaagagattgggtgggtttaccgataaggaaatcagtgacgtttttgccaaatggaacaatggtgtcttggattccttcttggtcgaaattaccagagatattttgaaattcgacgacgtcgacggtaagccattagttgaaaaaatcatggatactgctggtcaaaagggtactggtaagtggactgccatcaacgccttggatttgggtatgccagttactttgattggtgaagctgtctttgcccgttgtctatctgctttgaagaacgagagaattagagcctccaaggtcttaccaggcccagaagttccaaaagacgccgtcaaggacagagaacaatttgtcgatgatttggaacaagctttgtatgcttccaagattatttcttacgctcaaggtttcatgttgatccgtgaagctgctgctacttatggctggaaactaaacaaccctgccatcgctttgatgtggagaggtggttgtatcattagatctgttttcttgggtcaaatcacaaaggcctacagagaagaaccagatttggaaaacttgttgttcaacaagttcttcgctgatgccgtcaccaaggctcaatctggttggagaaagtcaattgcgttggctaccacctacggtatcccaacaccagccttttccaccgctttgtctttctacgatgggtacagatctgaaagattgccagccaacttactacaagctcaacgtgactactttggtgctcacactttcagagtgttgccagaatgtgcttctgacaacttgccagtagacaaggatatccatatcaactggactggccacggtggtaatgtttcttcctctacataccaagcttaa

SEQ ID NO: 33 - DNA, 3211 bp, Unknown
  Gdh2
aatatcatccatgtcggactatcacgtgtttgattttcccggtaaggacctgcagagagaggaagtgatagatttgctagatcagcaagggtttattcccgacgatttgatcgaacaagaagtagattggttttataactcattgggtattgacgatttgttcttctcgagagaatctccccaattaatctcgaatatcatacattctttgtatgcttcaaagctagatttctttgcgaagtccaaattcaacggaattcagccaaggctattcagcattaaaaacaaaattataactaatgataatcatgccatctttatggaatctaatactggtgtcagcataagcgattctcagcaaaaaaactttaaatttgctagtgacgccgtcggaaacgatactttggagcatggtaaggataccatcaaaaaaaataggattgaaatggatgattcttgtccaccttatgaattagattccgaaattgatgaccttttcctggataacaagtctcaaaaaaactgcagattagtttctttttgggctccagaaagcgaattaaagctaacttttgtttatgagagtgtttaccctaatgatgatccagccggcgtagatatttcctctcaggatttgctgaaaggtgatattgaatcgattagtgataagaccatgtacaaagtttcgtcgaacgaaaataaaaaactatacggtctcttacttaagttggttaaagaaagagaaggtcctgtcattaagactactcgctccgtagaaaataaggatgaaattaggttattagtcgcttacaagcgattcaccactaagcgttattactctgctttgaactctttgttccactattacaagttgaaaccttctaagttctatttagagtcgtttaatgttaaggatgatgacatcattatcttttccgtttatttgaacgagaaccagcaattggaagatgttctacttcacgatgtggaggcagcattgaaacaggttgaaagagaagcttcattgctatacgctatcccaaacaattctttccatgaggtttaccagagacgtcaattctcgcccaaagaagctatatatgctcatattggtgctatattcattaaccattttgttaatcgtttaggctctgattatcaaaaccttttatctcaaatcaccattaagcgtaatgatactactcttttggagattgtagaaaacctaaaaagaaagttaagaaatgaaaccttaactcagcaaactattatcaacatcatgtcgaagcattacactataatttccaagttgtataaaaattttgctcaaattcactattatcataatagtactaaagatatggagaagacattatcttttcaaagactggaaaaagtggagccttttaagaatgaccaagagttcgaagcttacttgaataaattcattccaaatgattcacctgatttgttgatcctgaaaacactgaacatcttcaacaagtctattttgaagacaaatttctttattacaagaaaagtagcaatatcattcagattagatccttccctggtgatgacaaaattcgaatatccagagacaccctatggtatattttttgtcgttggtaatactttcaaagggttccatatcaggttcagagatatcgcaaggggcggtattcgtatagtctgttccaggaatcaggatatttatgatttgaattccaagaacgttattgatgagaactatcaattggcctctactcagcaacgtaaaaataaggatattccagagggtggctctaaaggtgtcatcttattgaacccaggattggtagaacatgaccagacatttgtcgccttttcccaatatgtggatgcaatgattgacattctaatcaacgatccattaaaggaaaactatgtcaaccttttaccaaaggaggaaatattattttttggcccagatgaaggaactgctggtttcgtggattgggcaactaaccatgctcgtgtgaggaactgcccatggtggaaatcatttttgactggaaaatccccatctttgggtggtattccccatgacgaatatggtatgacttctctgggtgttcgtgcttatgttaataaaatttacgaaactttaaacttgacaaattctactgtttacaaattccaaactggtggtccggatggtgatttgggatccaatgaaattcttttatcttcgccaaacgaatgttatttggcaattctggacggttcaggtgtcctgtgtgatcctaaaggtttagataaagatgaattatgccgcttggcacatgaaaggaaaatgatttccgatttcgacacttccaaattatcaaacaacggattttttgtttctgtggatgcaatggatatcatgctaccaaatggtacaattgtagctaacggcacaaccttcagaaacacctttcatactcaaattttcaaatttgtggatcatgtcgacatttttgttccatgcggtggtagaccaaactcaattactctaaataatctacattattttgttgacgaaaagactgggaaatgtaaaattccatatattgtggagggtgccaatctatttataacgcaacctgctaaaaatgctttggaggaacatggctgtattctgttcaaagatgcttctgcaaacaaaggtggtgtcacatcttcatcaatggaagtgttggcctcactagcgcttaacgataacgacttcgtgcacaaatttattggagatgttagtggtgagaggtctgcgttgtacaagtcgtacgttgtagaagtgcagtcaagaattcagaaaaatgctgaattagagtttggtcagttatggaatttgaatcaactaaatggaacccacatttcagaaatttcaaaccaattgtccttcactataaacaaattgaacgacgatctagttgcttctcaagagttgtggctcaatgatctaaaattaagaaactacctattgttggataaaataattccaaaaattctgattgatgttgctgggcctcagtccgtattggaaaacattccagagagctatttgaaagttcttctgtcgagttacttatcaagcacttttgtttaccagaacggtatcgatgttaacattggaaaattcttggaatttattggtgggttaaaaagagaagcggaggcaagtgcttga

SEQ ID NO: 34 - DNA, 2232 bp, Unknown
  Stb5
atggatggtcccaattttgcacatcaaggcgggagatcacaacgtactactgaattgtattcgtgcgcacgatgcagaaaattaaagaagaagtgtggtaaacaaataccgacatgtgcaaactgcgataaaaatggggcacactgttcatatccaggtagagccccaagacgtaccaagaaggagttagcggatgctatgctacgaggggaatatgttccagtgaaaaggaacaagaaggtaggaaaaagcccattgagcactaagagcatgccaaactcttctagtccgctatccgcaaatggcgctataactcccgggttttcgccttacgaaaacgatgatgcacataagatgaaacagctaaaaccgtcagatccaataaatcttgtcatgggggcaagtccaaattctagcgaaggtgtctcatcgctaatttcggtgctaacatcgctgaatgataattctaatccttcttcgcacttatcctctaatgaaaattccatgattccttcccgatcattgccagcttccgtgcagcaaagttcgacaacttcatcattcggaggatataacacgccttcaccactaattagcagtcatgtgcctgcgaacgcccaagccgtaccgctacaaaacaacaatcgcaatactagcaacggggataacggcagtaatgttaaccacgataataacaatggcagtaccaacacaccgcaattgagtcttaccccatatgcaaacaattcagcccccaatgggaaattcgattctgtgccggttgatgcatcctcgatcgaatttgaaactatgtcctgttgctttaaaggtggtagaacaacatcgtgggtcagagaggatggctcgttcaagtcaattgatagatccttactggacaggttcattgccgcatacttcaaacacaatcaccgtctatttcccatgattgataaaatagcattcctaaatgacgccgcgacaattactgatttcgaaaggttatatgacaacaaaaactaccctgacagctttgttttcaaagtatacatgatcatggctattggttgtacaactttacagcgtgctggtatggtttctcaggacgaagagtgtctgagtgaacatttggcttttttggccatgaaaaaatttcgtagtgttataattttacaagatatcgaaactgtacgatgcctattgttgttgggtatttattcgttttttgagccaaagggctcctcgtcatggacaattagtggtatcatcatgcgattgaccataggattaggtttaaatagagagttaactgccaaaaaactcaagagcatgtctgctttagaagcagaggcaagatatagagtgttttggagtgcttactgctttgaaaggctagtatgcacctcgttgggccgtatatccgggatcgacgacgaagacatcactgtgccactaccgagggcgttgtatgtggatgaaagagacgatttagagatgaccaagttgatgatatcattaaggaaaatgggcggtcgcatttataaacaagtccactctgtaagtgcagggcgacaaaagttaaccatcgaacaaaagcaggaaatcataagtggattacgcaaggagctagacgaaatttattctcgagaatcagaaagaaggaaactgaaaaaatctcaaatggatcaggtggagagggagaacaattctactacaaatgtaatatccttccatagttctgagatttggctagcaatgagatactcccagttgcaaatcttactatacagaccatctgcattgatgccaaaaccgcccattgactcactatccactctaggagagttttgcttgcaagcctggaaacatacttatacactgtacaagaagcggttattacccttgaactggataacccttttcagaacattaaccatttgtaacactatcttatactgtctttgccagtggagcatcgacctcattgaaagtaaaatcgaaattcaacagtgtgtggaaatactaagacatttcggtgaaagatggatttttgctatgagatgcgcggatgttttccaaaacattagcaacacaattctcgatattagtttaagccatggtaaagttcctaatatggaccaattaacaagagagttatttggcgccagcgattcatatcaagacatattagacgaaaacaatgttgacgtctcttgggttgataaacttgtatga